(19) United States
(12) Patent Application Publication
(10) Pub. No.: US 2021/0284948 A1
(43) Pub. Date: Sep. 16, 2021

(54) METHOD AND APPARATUS FOR AUTOMATED INDEPENDENT PARALLEL BATCH-PROCESSING OF CELLS

(71) Applicant: Celyad, Mont-Saint-Guibert (BE)

(72) Inventors: Diederik Hauwaerts, Mont-Saint-Guibert (BE); Stefano Baila, Mont-Saint-Guibert (BE); Valérie Steenwinckel, Mont-Saint-Guibert (BE); Benjamin Demoulin, Mont-Saint-Guibert (BE); Kenneth Galbraith, Victoria (AU); Richard Grant, Victoria (AU); Mark Rob, Victoria (AU)

(21) Appl. No.: 16/319,211

(22) PCT Filed: Jul. 21, 2017
(86) PCT No.: PCT/EP2017/068535

§ 371 Date: Jan. 18, 2019

(30) Foreign Application Priority Data
Jul. 21, 2016 (EP) 16180486.9
Dec. 9, 2016 (EP) 16203292.4

(51) Int. Cl. C12M 1/36; C12M 3/00; C12M 1/00

(52) U.S. Cl. CPC C12M 41/48; C12M 23/44; C12M 23/28; C12M 23/58; C12M 23/42

(57) ABSTRACT

A cell processing device is provided that is suitable for performing independent concurrent processing of a plurality of cell preparations, the cell processing device comprising: (I) a cell processing station; and (II) a plurality of cell processing modules, wherein the plurality of cell processing modules engage and communicate with the cell processing station; wherein each of the plurality of cell processing modules comprises discrete processing compartments defined within, the processing compartments comprising: (i) a reagent pack, the reagent pack comprising one or more reagent vessels; d (ii) one or more fluidic cartridges, each fluidic cartridge comprising one or more cell processing compartments and a cell incubation compartment, wherein each of the processing compartments is in fluid communication with each other. A fluidic cartridge is also provided as is a method of use of the cell processing device

### FIELD OF THE INVENTION

The present application relates to novel methods and apparatus for the automated batch processing of cellular samples. In particular, it relates to the parallel batch processing of advanced therapy medicinal products (ATMP), such as CAR-T cell therapy products.

### BACKGROUND

Over recent years there has been growing interest in the field of cell therapy. Cell therapy involves the introduction of cellular material, generally intact, living cells into a patient, for the treatment of a variety of illnesses including cancer, as well as in regenerative medicine.

Cell therapy involves the production of the cells used for treatment, otherwise known as advanced therapy medicinal products (ATMPs). To address potential risk of systemic immunological reactions and disease transmission, ATMPs are often derived from the patient's own cells in a process known as autologous cell therapy (ACT), or autologous regenerative therapy.

Autologous cell therapy in its broadest form involves the removal of suitable cellular material from a patient, selection and expansion of cells, optionally ex-vivo modification of the cells, followed by appropriate quality control measures prior to re-introduction of the processed cellular material back into the same patient. A relevant example of such a process is the production of Chimeric antigen receptor T-cell (CAR-T cell) therapy products for the treatment of cancer.

In a typical procedure to produce CAR-T cells for therapy, the cellular material is removed from the patient in the form of a bodily fluid, such a blood. T-cells are then isolated and modified such that they express receptors on the cell surface specific to the particular form of cancer from which the patient is suffering. On re-introduction to the patient, the cells have the ability to target and kill the cancer. In addition, the cells have the ability to proliferate, thereby amplifying the immune response. Autologous CAR-T cell therapy is a powerful new technique in the treatment of cancer that is providing dramatic improvements in response rates in some cases.

The cost of production of ATMPs generally remains high due to the complexity and strict handling processes that must be adopted. The workflow to produce the required modified cellular material involves isolation of the appropriate cell type, modification, concentration and/or expansion by culture prior to re-introduction. There is also a requirement for sterile conditions throughout and strict quality control procedures during and at the end of the process to ensure strict safety and regulatory requirements are met. In autologous procedures there is the additional requirement that each patient sample must maintain its integrity and any degree of cross-contamination with other patient samples must be avoided to prevent the patient being exposed to cells not wholly derived from the patient's own cells.

Historically, ATMPs have been produced via conventional open laboratory environments. Here, the sterility and the integrity of the patient samples is maintained by the use of so-called ‘clean room’ facilities where the risk cross-contamination of samples is diminished though the use of suitable operating conditions and equipment (such as laminar air flow cabinets and appropriate sterilisation techniques). This often necessitates that the facility operates in a serial, ‘one-after-the-other’, process. Efforts to increase throughput of the production process under these conditions can lead to a greater likelihood of cross contamination of samples. There are also logistical and safety difficulties in material handling, such as reagent handling/dosing, and in particular sample tracking, to be overcome.

An alternative to increasing throughput in a serial fashion is adopting a parallel approach to cell processing. To date, methods and systems for parallel processing of cellular samples make use of multiple separate instruments. However, these processes still operate under the general principle of ‘one sample per instrument’. The addition of extra equipment and staff to operate the facility inherently increases capacity however this approach also leads to a significant increase in capital expenditure and staffing costs. Furthermore, as a common method of reducing the risk of cross-contamination of individual patient samples is to physically separate samples, often to separate rooms, then this also results in an increased requirement for clean room space which is often at a premium in manufacturing facilities or healthcare environments. In addition, with this approach it is difficult to realise savings by centralizing common resources from each instrument as a shared facility, such as quality control systems, power supplies and input gas input manifolds.

Central processing of cellular preparations on integrated process systems has also been attempted with limited success. There have also been attempts at central processing with manual manipulations. Systems developed to date have been complex and have, as a result, encountered difficulties due to very high cost and the need to train sufficient numbers of operators. An example of such a facility is described in US 2009/0126285.

Parallel processing of cellular material for cell therapy has been described in WO 2016/012459. In this approach, samples are isolated in sterile compartments within disposable closed processing plates. The samples within each plate are processed, as one, through processing units located within a processing station. The processing station is adapted to have greater capacity for plates in processing units that take longer thereby ensuring that samples progress through the full process as efficiently as possible with minimal delay. While the equipment described in WO 2016/012459 addresses the problem of increasing capacity in bulk processing systems it fails to address the need for entirely independent processing of individual samples in parallel. The system of WO 2016/012459 therefore fails to provide the flexibility of processing samples in a truly independent parallel fashion while realising the same cost savings in terms of reduced capital expenditure and reduced requirements for clean room space.

A need therefore remains for a system that provides for the automated independent parallel processing of cellular preparations, in particular cellular preparations for cell therapy, which does not suffer from the deficiencies of known systems.

### SUMMARY

It is an object of the present invention to overcome or alleviate at least one of the above noted drawbacks of prior art systems or to at least provide a useful alternative to related art systems.

A first aspect of the invention provides a cell processing device suitable for performing independent concurrent processing of a plurality of cell preparations, the cell processing device comprising:
(I) a cell processing station; and
(II) a plurality of cell processing modules,
   wherein the plurality of cell processing modules engage and communicate with the cell processing station;
   wherein each of the plurality of cell processing modules comprises discrete processing compartments defined within, the processing compartments comprising:
i. a reagent pack, the reagent pack comprising one or more reagent vessels;
ii. one or more fluidic cartridges, each fluidic cartridge comprising one or more cell processing compartments and a cell incubation compartment,
   wherein each of the processing compartments is in fluid communication with each other.

Suitably, the device is configured such that each of the plurality of cell preparations are physically and/or temporally separated throughout processing.

In an embodiment, each of the plurality of cell processing modules is adapted to process a single cell preparation at once. Suitably, the cell processing module comprises a closed, substantially aseptic environment for cell processing. Typically, the closed, substantially aseptic environment is provided within the reagent pack and/or the fluidic cartridge.

In an embodiment, at least part of the cell processing module is a single-use component. Suitably, substantially all of the cell processing module is a single-use component. In embodiments, the single use component of the cell processing module is selected from the group consisting of: the fluidic cartridge; the reagent pack; and both the fluidic cartridge and the reagent pack (the cell processing unit).

In an embodiment, the cell processing station comprises centralised facilities that may be used by one or more of the plurality of cell processing modules. Suitably, the centralised facilities are selected from the group consisting of:
i. a platform chassis;
ii. a user interface display;
iii. software and operating system licenses;
iv. a central processing unit (CPU);
v. a main control system including embedded controller and Program Logic Controller (PLC);
vi. a power supply and power distribution system;
vii. thermal management equipment required for controlling the temperature of the cell processing module or parts thereof;
viii. an incubator gas mixture supply facility;
ix. one or more systems used for in situ measurement and/or testing; and
x. a centrifugation drive system.

In embodiments of the device according to the first aspect of the invention where the in situ measurement and/or testing is present in the centralised facilities of the cell processing station, they are selected from the group consisting of: cell counting; cell identification; purity; homogeneity; potency; characterisation; and means for quality control.

In an embodiment, the cell processing module is adapted to contain the cell preparation throughout cell processing.

In a further embodiment, each of the plurality of cell processing modules comprises one or more connectors suitable for connection with the centralised facilities on the cell processing system.

In embodiments, the one or more reagent vessels in the reagent pack are adapted to contain one or more non-cellular fluids required for cell processing. Suitably, the reagent pack comprises one or more removable connectors to connect the reagent pack to the fluidic cartridge. Typically, each fluidic cartridge is configured to contain and manipulate a single cell preparation throughout cell processing. In an embodiment, each cell processing modules comprises a single fluidic cartridge.

In an embodiment of the first aspect of the invention, the fluidic cartridge comprises at least one input port, a separation chamber, an activation chamber, a transduction chamber, a cell culture chamber, and at least one output port. Suitably, the fluidic cartridge further comprises elements selected from the group consisting of:
i. Filters, centrifuges and other active surfaces for separating the cell preparation into different components;
ii. Reaction vessels, flasks and beakers for incubating cells and introducing active reagents;
iii. Galleries, channels and fluidic circuits to direct the flow of fluids around, into and out of the fluidic cartridge.

In an embodiment, the fluidic cartridge further comprises at least one pump and at least one valve to at least partially control fluid flow, wherein fluid flow is selected from the group consisting of: flow through a fluid circuitry in the fluidic cartridge; flow into the fluidic cartridge; and flow out of the fluidic cartridge. Suitably, the at least one pump is selected from the group consisting of a positive displacement pump, a diaphragm, a plunger-style pump, impeller pumps, peristaltic pumps; and wherein the valve is selected from the group consisting of a diaphragm valve, a rotating valve; and a combination thereof.

In a second aspect of the present invention a fluidic cartridge is provided that is suitable for providing a closed aseptic environment for cell processing therein, wherein the fluidic cartridge comprises at least one input port, a separation chamber, an activation chamber, a transduction chamber, a cell culture chamber, and at least one output port.

In embodiments, the fluidic cartridge further comprises elements selected from the group consisting of:
i. Filters, centrifuges and other active surfaces for separating the cell preparation into different components;
ii. Reaction vessels, flasks and beakers for incubating cells and introducing active reagents; and
iii. Galleries, channels and fluidic circuits to direct the flow of fluids around, into and out of the fluidic cartridge.

In an embodiment, the fluidic cartridge further comprises at least one pump and at least one valve to at least partially control fluid flow, wherein fluid flow is comprises flow of fluid through a fluid circuitry in the fluidic cartridge, flow into the fluidic cartridge, and flow out of the fluidic cartridge. Suitably, the at least one pump is selected from the group consisting of: a positive displacement pump, a diaphragm, a plunger-style pump, impeller pumps, peristaltic pumps and a combination thereof; and wherein the valve is selected from the group consisting of a diaphragm valve, a rotating valve; and a combination thereof.

In an embodiment of the second aspect of the invention the fluidic cartridge is a single-use component.

A third aspect of the present invention provides a cell processing unit, wherein the cell processing unit comprises one or more fluidic cartridges and a reagent pack, wherein the fluidic cartridge comprises at least one input port, a separation chamber, an activation chamber, a transduction chamber, a cell culture chamber, and at least one output port, and wherein the reagent pack comprises one or more reagent vessels, wherein the one or more reagent vessels in the reagent pack are adapted to contain one or more non-cellular fluids required for cell processing. Suitably, the fluidic cartridge and the reagent pack are an integrated single entity. Typically, the cell processing unit is a single-use component.

In embodiments of the third aspect of the invention, the fluidic cartridge is the fluidic cartridge of the second aspect of the invention.

In a fourth aspect of the present invention, a cell processing module is provided that is suitable for integration with a cell processing station, wherein the cell processing module comprises:
a cell processing unit, the cell processing unit comprising a fluidic cartridge and a reagent pack, wherein the fluidic cartridge comprises at least one input port, a separation chamber, an activation chamber, a transduction chamber, a cell culture chamber, and at least one output port, and wherein the reagent pack comprises one or more reagent vessels, wherein the one or more reagent vessels in the reagent pack are adapted to contain one or more non-cellular fluids required for cell processing; and
a means to retain and engage the fluidic cartridge and the reagent pack such that fluidic connection points of the fluidic cartridge and the reagent pack couple to provide fluid communication between the fluidic cartridge and the reagent pack.

In embodiments of the fourth aspect of the invention, the cell processing module of claim 31, wherein the cell processing module further comprises one or more of:
at least one connector for connection of the cell processing module or components thereof to the cell processing station;
at least one sensor;
an incubator enclosure to provide a temperature controlled environment to the cell processing module;
a heater pad and controller to provide thermal energy to the cell processing module;
one or more mechanical actuators housed within the cell processing module to actuate elements within the fluidic cartridge;
one or more mechanical actuators housed within the cell processing module to actuate elements within the reagent pack;
a centrifugal facility.

Suitably, the at least one sensor is selected from the group consisting of thermocouples/thermistors to measure temperature; pressure transducers to measure fluid pressure; flow meters to measure fluid flow rate; and biosensors as required by the process.

In an embodiment, the cell processing unit is a single use component.

In further embodiments, the fluidic cartridge is the fluidic cartridge of the second aspect of the invention; and/or the cell processing unit is the cell processing unit of the third aspect of the invention.

A fifth aspect of the invention provides a method for performing independent concurrent processing of at least two cell preparations, wherein the method comprises:
1) Providing a cell preparation;
2) Providing a fluidic cartridge, wherein the fluidic cartridge comprises one or more cell processing compartments and a cell incubation compartment;
3) Placing the cell preparation in the fluidic cartridge;
4) Assembling a cell processing module comprising the fluidic cartridge and a reagent pack;
5) Engaging the cell processing module with an available receiving point on a cell processing station;
6) Operating the cell processing station to perform automated cell processing of the cell preparation to provide a processed cell preparation;
7) Repeating steps 1 to 6 with a further cell preparation so that at least two cell preparations are being processed independently and concurrently on the cell processing station;
8) Optionally repeating step 7 until all available receiving points on the cell processing station are occupied with cell processing modules;
9) Removing the cell processing module from the cell processing station when automated cell processing of that cell preparation is complete.
10) Repeat step 9 for each cell processing module on the cell processing station;
   wherein steps (3), (4) and (5) of the method are performed in any order between step (2) and step (6).

In embodiments of the fifth aspect of the invention, the cell processing of each cell preparation is spatially and/or temporally separated from each other cell preparation.

In further embodiments, the method is performed in a closed, aseptic environment. Suitably, the closed, aseptic environment is within one or more of the fluidic cartridge and the reagent pack.

In an embodiment, the cell preparation is further processed prior to removal of the cell processing module from the cell processing station. Suitably, the further processing is selected from the group consisting of freezing for cryopreservation and formulating into a composition comprising the processed cell preparation.

In embodiments of the fifth aspect of the invention, the automated cell processing comprises one or more steps selected from the group consisting of:
a. Selection of the cells;
b. Enrichment of the cells;
c. Activation of the selected cells of step (a) and/or the enriched cells of step (b);
d. Genetic modification of the cells to provide genetically modified cells;
e. Expansion of the genetically modified cells of step (d) to provide expanded cells;
f. Washing of the expanded cells of step (e);
g. Concentration of the expanded cells of step (e);
h. Formulation of the expanded cells of step (e) to provide a formulation of cells, wherein the formulation of cells is for preservation and/or direct injection.

In an embodiment, the cells are T cells. Suitably, the genetic modification in step (d) is genetic modification with a chimeric antigen receptor (CAR). Typically, the CAR is selected from the group consisting of: NKG2D CAR and B7H6 CAR.

In further embodiments, the method is performed on a cell processing device according to first aspect of the invention; the fluidic cartridge is the fluidic cartridge of the second aspect of the invention; and/or the cell processing module is the cell processing module of the fourth aspect of the invention.

A sixth aspect of the invention provides a computer device comprising a processor, and a memory encoding one or more non-neural network programs coupled to the processor, wherein said programs cause the processor to perform a method according to the fifth aspect of the invention.

### BRIEF DESCRIPTION OF THE DRAWINGS

FIG. 1 shows a schematic representation of the cell processing device according to the present invention with four cell processing modules engaged.

FIG. 2a shows a computer-generated representation of an embodiment of the cell processing device according to the present invention that is adapted to accommodate two cell processing modules that are mounted on trolleys. One cell processing module is shown disengaged from the cell processing device so that the fluidic cartridge and reagent pack can be seen.

FIG. 2b shows a schematic representation of an embodiment of the cell processing device according to the present invention that is adapted to accommodate four cell processing modules that are not mounted on trolleys. One cell processing module is shown disengaged from the cell processing device so that the fluidic cartridge and reagent pack can be seen.

FIG. 2c shows a schematic representation of an embodiment of a cell processing module that is not mounted on a trolley.

FIG. 3 shows an embodiment of a cell processing unit according to the invention comprising a fluidic cartridge and a reagent pack in assembled form.

FIG. 4 shows a cell processing unit comprising a fluidic cartridge and a reagent pack in exploded form.

FIG. 5 shows cross-sectional perspective view of an embodiment of the activation, transduction and expansion areas within the fluidic cartridge.

FIG. 6 shows an embodiment of how patient-derived material can be connected to and included in a CPM that already comprises a reagent pack and fluidic cartridge.

FIG. 7 shows an example of a basic process flow for cell processing within the device according to the present invention.

FIG. 8 shows an example of a detailed process flow for cell processing within the device according to the present invention.

FIG. 9 shows an embodiment of a process flow for quality control and analytics in an automated process for the production of T-cells according to an embodiment of the present invention.

FIG. 10 shows expansion (FIG. 10a) and viability (FIG. 10b) data for T-cells produced on an embodiment of the device of the present invention.

### DETAILED DESCRIPTION

Prior to setting forth the invention, a number of definitions are provided solely to assist in the understanding of the invention. All references cited herein are incorporated by reference in their entirety. Unless otherwise defined, all technical and scientific terms used herein have the same meaning as commonly understood by one of ordinary skill in the art to which this invention belongs.

The present invention will be described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes.

As used herein, the term ‘comprising’ means any of the recited elements are necessarily included and other elements may optionally be included as well. ‘Consisting essentially of’ means any recited elements are necessarily included, elements that would materially affect the basic and novel characteristics of the listed elements are excluded, and other elements may optionally be included. ‘Consisting of’ means that all elements other than those listed are excluded. Embodiments defined by each of these terms are within the scope of this invention.

Where an indefinite or definite article is used when referring to a singular noun e.g. “a” or “an”, “the”, this includes a plural of that noun unless something else is specifically stated. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

Unless otherwise indicated, the practice of the present invention employs conventional techniques of chemistry, molecular biology, microbiology, recombinant DNA technology, and chemical methods, which are within the capabilities of a person of ordinary skill in the art. Such techniques are also explained in the literature, for example, M. R. Green, J. Sambrook, 2012, Molecular Cloning: A Laboratory Manual, Fourth Edition, Books 1-3, Cold Spring Harbor Laboratory Press, Cold Spring Harbor, N.Y.; Ausubel, F. M. et al. (1995 and periodic supplements; Current Protocols in Molecular Biology, ch. 9, 13, and 16, John Wiley & Sons, New York, N. Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridisation: Principles and Practice, Oxford University Press; M. J. Gait (Editor), 1984, Oligonucleotide Synthesis: A Practical Approach, IRL Press; and D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

The term ‘cell’ as used herein typically refers to eukaryotic cells, more particularly mammalian cells, most particularly human cells.

The term ‘independent’ as used herein in reference to the concurrent, simultaneous or parallel processing of cellular samples, is defined as each samples being processed in a manner that is both spatially (physically) and temporally (time) separate from the processing of any other sample being processed or to be processed on the same device. To avoid doubt, the term ‘independent’, in particular when applied to temporal independence, includes interaction of samples to the extent that if there is a conflict in timing between the need for more than one sample to use a shared facility then the processing of one or more samples may be delayed or re-routed to accommodate this.

The phrase “in fluid communication” as used herein in reference to processing compartments may refer to direct fluid communication (i.e. both compartments are immediately adjacent) or indirect communication (fluid may need to pass through e.g. a vessel, tube or other compartment before it reaches the processing compartment), as long as the fluid is not permanently impeded from flowing between compartments. The flow may be bidirectional, unidirectional, or in more than two directions.

The term ‘autologous’ as used herein refers to the same individual. The term ‘allogeneic’ as used herein refers to the same species, but a different individual.

The term ‘apheresis’ as used herein is defined as a medical technology in which the blood of a subject is passed through an apparatus that separates out one particular constituent and returns the remainder to the circulation. The apheresis procedure is therefore an extracorporeal process.

As used herein the term ‘PBMC’ is a peripheral blood mononuclear cell. This cell type refers to any peripheral blood cell having a round nucleus. These cells consist of lymphocytes (T cells, B cells, NK cells) and monocytes.

The term ‘T cells’ as used herein refers to lymphocyte cells having a T-cell receptor on its cell surface. The term refers to T cells of all types (effector, helper, cytotoxic or killer, memory, regulatory or suppressor, natural killer, mucosal associated invariant and gamma delta) as well as T cell subsets, and/or T cell progenitors. Note that for some allogeneic applications, T cells may be made T-cell receptor deficient, but these TCR-deficient T cells remain envisaged as T cells herein.

It is an object of the invention to provide systems and methods of use of those systems to increase the flexibility and throughput of production of cellular products, in particular advanced therapy medicinal products (ATMPs). An example of an ATMP is a CAR-T cell therapy product.

The systems according to the present invention perform a range of manufacturing process steps on biological material or samples removed from a patient, such as blood. All processes are performed within a closed, sterile environment. The device is intended to process materials or samples from multiple patients concurrently with no cross-contamination between samples. The processing of a given sample is in parallel with, and in a manner that is entirely independent of, other samples previously or subsequently processed in the device.

The systems according to the present invention have capacity to process a plurality of patient samples in an independent manner concurrently on one device. The systems may be automated to minimize operator interventions and to improve the accuracy of sample and reagent handling, and to minimise error in sample tracking.

The use of such systems for concurrent processing significantly reduces waiting time: in typical devices that can process multiple cell batches, these cell batches need to be loaded simultaneously, and no new batches can be added until the process is completed. This causes delays both in starting operation of the cell manufacturing system (sufficient batches of cells need to be present before operating the device, as otherwise it is not maximally used), as well as delays in starting processing of a new cell batch (if a typical cell manufacturing device is operational, the operation needs to be terminated before a new batch can be loaded). As cell therapies are particularly envisaged for very ill patients who require urgent treatment, such reduction of delays is hugely beneficial.

In more detail, the system of the present invention comprises a cell processing station (CPS) that houses one or more cell processing modules (CPMs). Each CPM represents a closed system for a single cellular sample for processing that, once assembled, is isolated from contamination from the external environment and from the other CPMs in the system.

Each CPM comprises one or more fluidic cartridges (FCs) in which the cellular sample is loaded. Typically, each CPM comprises a single FC. The FC may be removable from the CPM or it may be permanently attached to the CPM and samples are loaded directly therein. Suitably the FC is removable from the CPM. Suitably the FC is provided in a substantially sterile or aseptic condition prior to loading of the cellular sample to prevent contamination of the sample to be loaded. Typically, the FC is a single-use cartridge that is provided new in an aseptic state for each new sample. Suitably, the FC is disposed of after the required cellular processing is complete and is not re-used.

Each CPM further comprises one or more reagent packs (RPs). The RP contains the various fluids required for cellular processing of the sample contained within the corresponding CPM. The RP may be removable from the CPM or it may be permanently attached to the CPM and reagents are loaded directly therein. Suitably the RP is removable from the CPM. Suitably the RP is provided in a substantially aseptic condition prior to loading of the reagents. The RP may be provided in a clean, sterilised condition to prevent contamination with the reagents to be loaded. Typically, the RP is a single-use cartridge that is provided new in an aseptic state for each new sample. Suitably, the RP is disposed of after the required cellular processing is complete and is not re-used.

A more detailed description of specific embodiments of each of the CPS, CPM, FC and RP are provided below:

### Cell Processing Station (CPS)

The CPS provides a central support platform in which one or more CPMs can be removably installed. The CPS may accommodate one or more CPMs at the same time. The CPS may be adapted to accommodate the number of CPMs desired by the facility in which it was operated. Typically, the CPS would have capacity to accommodate at least two CPMs at the same time. Suitably, the CPS would have capacity to accommodate three, four, five, six, seven, eight, nine or ten or more CPMs at the same time. In an embodiment, the CPS can accommodate four CPMs at the same time.

The CPS may further comprise one or more common or shared facilities (hereinafter ‘platform facilities’) that may be used by at least two installed CPMs. In embodiments, platform facilities may include a common power supply, control system and user interface, central incubator and refrigeration facilities, and platform QC systems.

Typical device features and technologies that are provided in the CPS that may be shared by the one or more CPMs include:
platform chassis;
user interface display;
software and operating system licenses
a central processing unit (CPU);
main control system including embedded controller and Program Logic Controller (PLC);
power suppliers and power distribution systems;
thermal management equipment required for heating and cooling, including freezing for e.g. cryopreservation;
systems used for in situ measurement potentially including cell counting, cell identification, potency, cell purity (or characterisation/measurement of impurities), homogeneity, cell characterisation, endotoxin detection, safety monitoring (e.g. by detection of viral copy number or replication competent retrovirus), mycoplasma detection;
centrifugation drive system.

In some cases, the use of facilities shared by multiple CPMs may reduce the redundancy of facilities used for only a part or a portion of each cellular process. An example of this may be a spectrophotometer used for quality control purposes only at set points within the cellular processing, or a centralised user control panel. Alternatively, shared facilities may provide more accurate or more consistent provision of services such as mixed temperature controlled supply gases or power.

The approach of the present invention is feasible due to the considerable portion of the overall manufacturing time that the cells spend in incubation operations; during incubation most of the fluid handling and cell handling technologies are inactive. Therefore, suitable moderate staggering of batch start times enables improved utilization of expensive technologies by sharing these technologies between cell processing modules.

In embodiments of the present invention, the CPS may further comprise one or more of the following:
An incubator gas mixture supply facility plumbed to each of the one or more CPMs loaded in the platform. A common incubator has the benefit of saving costs by having only one incubator supply facility and gas mixture control system along with providing a more thermally and stoichiometrically stable incubation environment for each of the one or more CPMs and the FCs contained therein. Individual CPM incubator enclosures could be integrated to become a single CPU incubator enclosure facility whereby the CPM closure and support structure for the FC could receive the FC directly into the common incubator enclosure.
A single user interface point for the control systems, monitoring and user operation procedure for each of the CPMs and certain common platform facilities. The user interface could also comprise of a graphical user interface displayed on an interactive touch screen interfacing with a programmable logic controller (PLC) environment and hardware;
Power supply infrastructure and distribution network to the variety of platform and CPM electrical components.
A quality control system to monitor mid-process the key cellular parameters and quality of each CPM and sample contained therein. The benefit of having a QC system as a platform facility is the cost savings by sharing an expensive piece of equipment between the CPMs given that it has relatively low utilisation within the overall process.

In an embodiment, the QC system comprises monitoring the status of the sample by one or more of cell counting, cell viability, cell characterisation or other cell/biomonitoring through the use of analytical equipment such as flow cytometers, optical cell counters. By way of non-limiting examples, the QC system may comprise the monitoring of cell counting and viability by microscopy, fluorescence, trypan blue, or flow cytometry; the monitoring of cell identity, purity, or homogeneity by flow cytometry or other methods; the detection of endotoxins (such as by a LAL test); monitoring of safety (e.g. by PCR of Viral Copy Number (VCN) and/or detection of Replication Competent Retrovirus (RCR-test)); Myclopasma detection (e.g. by PCR or Gram staining); monitoring of potency (e.g. by co-culture with cell lines).

Sharing of QC facilities between CPMs could be achieved by positioning the QC system at the datum position at each CPM by means of robotic or other mechanical means, or transmitting the output from each CPM QC datum location to the platform QC system by optical, electronic or other transmission means, or other methods in which the QC device can be shared between CPMs. It will be noted that the QC process could occur post process and outside the CPU itself. Similarly it will be noted that each CPM could have its own individual QC system.
An electroporation, electrofusion or a transfection device for electroporation, fusion or transfection of cells, respectively. Similarly, it will be noted that each CPM could have its own individual electroporation, electrofusion or transfection system. Also envisaged herein are the use of sonoporation, magnetofection or a gene gun to deliver nucleic acids into cells.
A refrigeration supply facility plumbed to the individual CPM refrigeration enclosures to provide the requisite temperature environment for the various reagents used during the process. It will be noted that the individual CPM refrigeration enclosures could be integrated to become a single CPS refrigeration enclosure facility whereby the CPM closure and support structure for the RP could receive the RP directly into the common refrigeration enclosure.

The system may further comprise a preservation means for preservation of the obtained cells. According to specific embodiments, the preservation means may be cryopreservation means. Particularly, the cells can be formulated for cryopreservation, provided in suitable containers (e.g. bags), and the cells in their containers are immediately cooled to the desired temperature, typically frozen. Typically, cryopreservation occurs at temperatures of −20° C. or below, −60° C. or below, −80° C. or below, or −196° C. or below. According to such embodiments, the CPS will be equipped with suitable freezing supply facilities. The process of preservation or cryopreservation may be achieved without the need for human intervention leading to advantages in terms of no need for personnel (this also implies that the cellular processing methods can be started at any time, as there is no need to match the end time of the process with the availability of a skilled operator), no time delay (i.e. immediate cryopreservation leading to preservation of product in optimal condition). Although the cells can in principle be stored in the system this way, it is typically envisaged that, once appropriately frozen, the bags can be transferred to a storage space more suited for longer-term storage, and the preservation means in the system are freed up for preserving new batches of cells.

According to particular embodiments, preservation means or a preservation chamber for preservation of the obtained cells is included within the system. Such preservation means or preservation chamber typically is suitable for cryopreservation and thus be capable of freezing the suitably formulated cells. Typically, the preservation chamber is intended for the initial freezing and possibly short-term storage. Although long-term storage may be feasible in the system, it is particularly envisaged that this will take place in another location, so the system is free for preservation of more batches.
A heating supply facility plumbed to the individual CPM heating enclosures to provide the requisite temperature environment for the various reagents used during the process. It will be noted that the individual CPM heating enclosures could be integrated to become a single CPS heating enclosure facility whereby the CPM closure and support structure for the RP could receive the RP directly into the common heating enclosure.

Typically, the heating supply facility is aimed at preserving the cells at a suitable temperature, particularly 37° C. or higher, 35° C. or higher, 32° C. or higher, 30° C. or higher, 25° C. or higher. However, the heating supply may for instance also be used to provide the correct temperature for QC-based methods, and in some of those embodiments, higher temperatures may be required (e.g. PCR-based methods require temperature cycles of between 94-98° C., 48-72° C., 68-72° C., depending on the enzymes that are used).

### Cell Processing Module (CPM)

The CPM is a discrete module that may be removably attached to, docked, integrated with or installed on the CPS. The CPM receives the FC and RP for a specific patient and cellular process. One or more CPMs can be installed in a CPS platform at once enabling multiple patient samples to be processed simultaneously. There is no limitation on the timing of installing or removing a second or further CPM into the CPS depending on when one or more earlier CPMs were installed. Suitably any new CPM is installed at a time dictated by the needs of the operator and the CPS will sequence the process start and subsequent operations in the most time efficient manner for all processes then running concurrently.

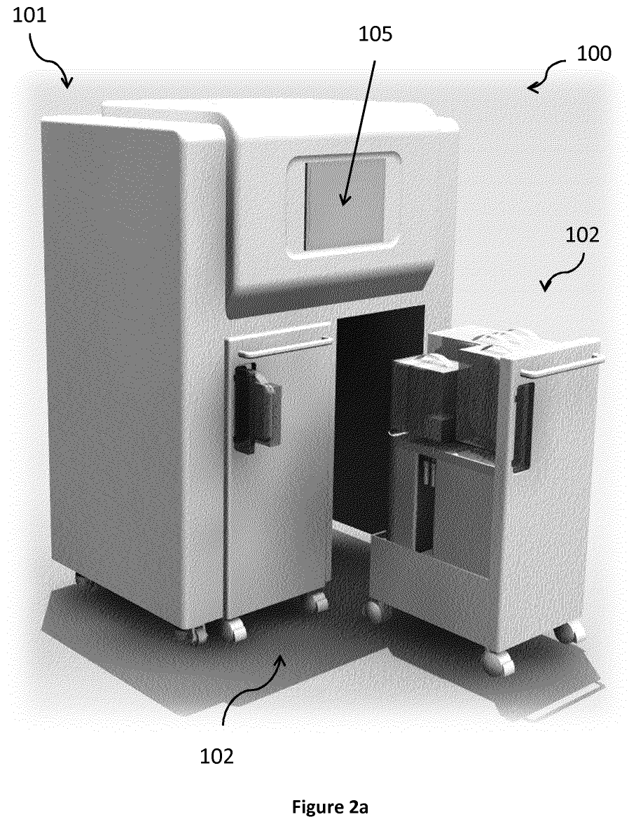

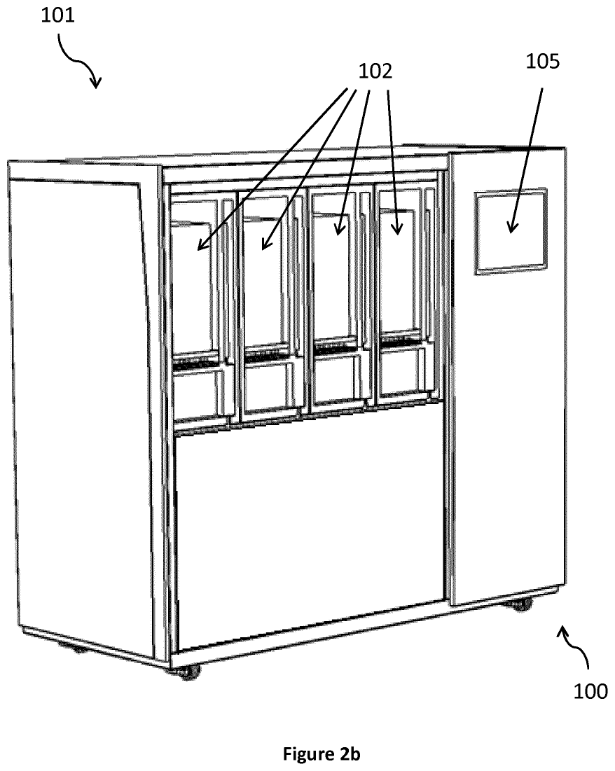

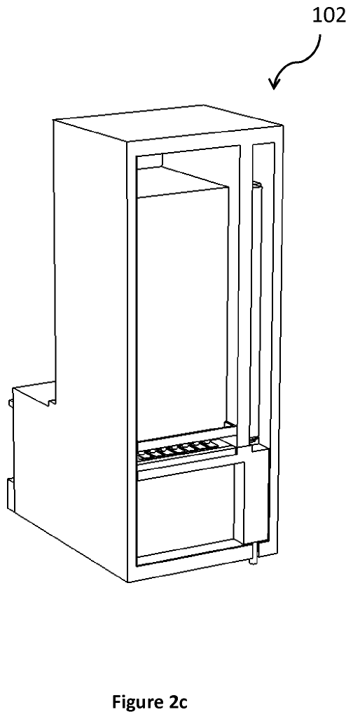

The CPM may be any size suitable for the cell processing for which it is envisaged. In an embodiment, the CPM is located on a trolley that may be manoeuvred around the production facility collecting the required components for the process, for example, components that make up the reagent pack and the fluidic cartridge containing the cell sample for processing, prior to installation in the CPS. An illustration of such an embodiment is given in FIG. 2a.

Large dose cell therapies require volumes of media and supporting reagents that when incorporated in to a reagent pack can result in an overall disposable weight that may be too high to carry safely. Adapting the CPM to be mounted on a trolley removes the need to have the reagent storage facilities in near proximity to the CPS reducing cost of installation and potential operator harm.

Mounting the CPM on wheels or other means of transport (such as in a trolley) also allows other advantages: the docking of the CPM to the CPS can be guided (e.g. by means of rails) and can be automated or semi-automated (e.g. with a brake system to avoid brusque coupling of the CPM to the CPS). A level tool or check for correct docking can be incorporated so that the CPS can display a positive or error message accordingly.

In an embodiment, the trolley could be used to collect the cell sample directly from the patient at the bedside. In embodiments the trolley is adapted to be able to dock or install into the CPS being appropriately wheeled into position.

Despite the advantages of having the CPM in a form that can be easily moved around (e.g. by the addition of wheels), this is not a prerequisite, and other designs can be considered (e.g. to save space). FIG. 2b shows an example of a CPS with four CPMs that can be manually loaded. FIG. 2c shows the CPM of this embodiment in more detail.

In embodiments, the CPM comprises one or more of:
A means to engage the FC and/or the RP such that the mating fluidic connection points of the FC and/or the RP couple, once the FC and/or the RP are enclosed within their respective enclosures. Such a means of engagement suitably does not compromise the thermal integrity of the respective enclosures. One possible method of engaging the FC and RP is the translation of either or both the FC and RP support structure to bring the two entities together. This could comprise of a lifting mechanism attached to either enclosure that is either a user operated or automatically initiated action. It will be noted that the engagement of the FC and RP could alternatively occur outside the machine, and furthermore be a process performed by the operator. It will also be noted that the engagement means may not be required if the FC and RP are an integrated entity.
A range of connections to the required CPS platform facilities, including electrical and data connections to the power supply, control system and user interface, plumbing to the incubator and refrigeration facilities, physical connections to the platform QC systems. It will be noted that another embodiment could see each individual CPM having their own dedicated facilities.
A range of sensors provided to the FC and reagents pack as required by the process to monitor a variety of different parameters. These sensors could include thermocouples or thermistors to measure temperature, pressure transducers to measure fluid pressure, flow meters to measure fluid flow rate, biosensors such as glucose monitors, oxygen sensors and other sensors as required by the process. Furthermore the sensors could provide a closed feedback control loop to optimise the variables within the various stages of the process.

In embodiments, the CPM comprises an incubator enclosure to provide the RP with a temperature controlled environment during the process. Suitably the enclosure provides a refrigerated (i.e. below ambient temperature) environment. Typically the temperature maintained within the refrigerated enclosure is less than about 15° C. Suitably the temperature is less than about 10° C., 8° C., 6° C., 4° C., 2° C., 0° C. In an embodiment the enclosure is refrigerated to provide an environment of about 4° C. The benefits of a refrigerated enclosure is providing the temperature-sensitive reagents a suitable environment to survive during the entire process and thus eliminating additional operator effort loading time and temperature sensitive reagents mid process and potentially holding up or disturbing the process. In an embodiment, the refrigerated enclosure is a sealed and insulated compartment within the CPM with a closure and support structure where the RP is inserted, retained and sealed. In an alternative embodiment, the refrigerated enclosure is a smaller compartment that provides only part of the RP with refrigerated facilities.

In an embodiment, the CPM may further comprise a heater pad and controller to provide additional thermal energy to the FC and/or reagent pack. Suitably the heater pad provides heat to the FC only. The benefit of having the heater pad in addition to the incubator enclosure is the means to rapidly heat the reagent fluids prior to, or on entering into, the FC which reduces any delay in raising the temperature of the reagent fluids via ambient heat. Similarly the heater pad provides more fine control over the temperature of the reagent fluids within the incubator. In embodiments, the heater pad is of any form suitable for providing the required heating characteristics. Suitably the heater pad is a Peltier thermal component, silicon based thermal wafers, electrically heated metal blocks or other means to provide heat energy. In embodiments, the heater pad replaces the incubator enclosure as the primary heat source for the process. Typically, the heating pad is configured for maintaining suitable temperatures in cell processing, particularly 37° C. or higher, 35° C. or higher, 32° C. or higher, 30° C. or higher, 25° C. or higher. However, the same or a different heating pad may also be used to provide the correct temperature for QC-based methods, and in some of those embodiments, higher temperatures may be required (e.g. PCR-based methods require temperature cycles of between 94-98° C., 48-72° C., 68-72° C., depending on the enzymes that are used).

In embodiments, the CPM further comprises mechanical actuators housed within the CPM and actuate elements within the FC such as pumps, valves or other dynamic components. For instance, a diaphragm pump can be used. This pumping technology utilises an oscillating diaphragm driven by either air pressure or mechanically to create a change in the volume of a fluid chamber (below the diaphragm) with each oscillation much like a piston in a cylinder. This pumping approach mimics the hearts action and is therefore believed to be the most natural and gentle on the cells. The use of this technology provides a tube free solution that is low in cost to manufacture and easy to assemble. To actuate the diaphragm in an intake and exhaust stroke, a pump motor in the CPM may be used on a crank type arrangement. Similarly, the pump may be driven pneumatically from a pressure and vacuum supply. Alternatively, or additionally, a valve diaphragm can be used, utilising an elastomeric diaphragm forced inward to create a fluidic seal against a weir stopping fluid flow when activated. Another dynamic component that is envisaged is a precision fluid dosing unit that can control the repeated dispensing of a highly accurate, small amounts of fluid. The benefit of the mechanical actuators being housing within the CPM is that it reduces the cost and complexity of the FC, particularly when the FC is a disposable, single-use component as only the lower cost pump and valve heads would be disposed of post-processing and the actuation means can be retained and used again.

In embodiments, the CPM comprises a centrifugal facility in which the FC can employ as part of the separation, activation, transduction, expansion or other stages of the process.

### Fluidic Cartridge (FC)

The FC is a component part of an individual CPM that contains and manipulates the cells throughout cellular processing. Each CPM may comprise any number of fluidic cartridges that is appropriate for the cellular process being conducted. Typically, each CPM comprises one or more FCs. In an embodiment, each CPM comprises one FC. The one or more FC, when fitted in the CPM, provide a closed structure sealed from cross contamination from other samples and the environment.

To maintain a sterile, aseptic condition, the FC may be used new in a sterile condition and disposed of after a single use (i.e. a single cell processing procedure). Alternatively, the FC may be suitably treated after use to return it to a sterile aseptic condition prior to repeat use.

The FC houses the cellular material and provides elements that may be used for the different steps of the process. In embodiments of the present invention these elements include:
Filters, centrifuges or other active surfaces for separating patient material (Separation).
Reaction vessels, flasks or beakers for incubating cells and introducing active reagents (Activation, Transduction, Expansion);
One or more vessels for waste disposal;
Galleries, channels and fluidic circuits to direct the flow of fluids from one region of the FC to another.

In embodiments, the FC also contains pumps and valves required to move the fluids and cells to the different positions within the FC assigned for various steps of the process. Suitably the type of pump used is a positive displacement pump such as a diaphragm or plunger-style pump although it is contemplated that any suitable pump type could be used. Similarly, the valve may suitably be a diaphragm or rotating valve, although any suitable valve type could be used.

The pumps and valves also enable fluids from the RP to be moved into the FC as required during the process. These fluids could include patient input material, reagent media, active reagents and/or other required fluids.

In embodiments, the FC also contains fluidic connection points to enable fluid communication between the FC and the RP to allow the two to be manufactured, supplied and loaded as separate items yet provide a closed system once installed in the CPM. The connection needs to maintain the sterile integrity of the FC throughout the process, and this can be achieved using, for example, tearaway, hermetically sealed or other sterile or aseptic connectors to fluidically connect the two items.

At least some of the fluidic circuits in the fluidic cartridge will transport fluids containing cells, possibly cells that have been cultured for a while. As these cells may tend to clump together, according to particular embodiments, the fluidic circuits that direct the flow of fluids in the reagent pack are designed to disrupt cellular clumping when the cellular product flows through. This can be achieved by changes in diameters of the tubing, and incorporating sharp turns (including 90° turns). This way, the flow of the cellular fluid is not linear, and will mimic the effects achieved by e.g. up and down pipetting, but without the need for any human intervention.

In embodiments of the present system, the RP could be integrated into the FC to provide the RP and the FC as a single unitary component. Such an approach may reduce operator effort, waste output or other benefits provided by an integrated combination.

In embodiments, the construction of the FC is a moulded and/or welded substantially rigid polymer assembly with elastomeric overmoulded regions with pump or valve features. There may be discrete filter elements encapsulated between moulded parts. In certain embodiments, the design and layout of the FC could use moulded-in fluid galleries to guide the fluids through the steps of the process with an integral diaphragm pump providing the motive force to move the appropriate fluid. The construction of the FC could alternatively comprise of a flexible tube network with discrete fluidic components such as impeller pumps, peristaltic pumps, filters or centrifuges held in a rigid frame. As a further alternative, the construction of the FC could be a combination of an integral moulded assembly with flexible tubular sections with discrete fluidic elements.

### Reagents Pack (RP)

The RP is a patient specific consumable product that contains the various non-cellular fluids required for the process. The fluids may be liquids, aqueous solutions, gaseous mixtures, gels, lyophilised material suspensions or other fluidic compositions. Suitably, the fluids may be patient input material, bulk media, active ingredients and other reagents as required. Suitably, reagents could include: patient whole blood, apheresis, isolated patient cellular material, cell growth media such as X Vivo, cell separation media such as Ficoll, maturation agents, cellular activators or inhibitors, cytokines, enzymes, antibodies or similar proteins, proteins, peptides, viruses or viral vectors (e.g. retroviruses, lentiviruses, adenoviruses or vectors based thereon), transposons (e.g. sleeping beauty), other nucleic acids (e.g. mRNA, shRNA, siRNA, miRNA, naked DNA, plasmids, lncRNA, antisense oligonucleotides), synthetic molecules (e.g. PNA, LNA, stapled peptides), buffers, reconstitution media.

Typically, for practical reasons, a distinction will be made between the patient-derived material and the other material. For example, under suitable storage conditions, non-patient derived material can be pre-assembled in a RP. The patient-derived material will only be included in, or connected to, the RP, when the cellular processing needs to take place. Thus, according to certain embodiments, the RP containing only non-patient derived material can already be connected to a FC and/or be incorporated in a CPM, with the patient-derived material only be included later. The patient-derived material may be incorporated in, or connected to, the RP.

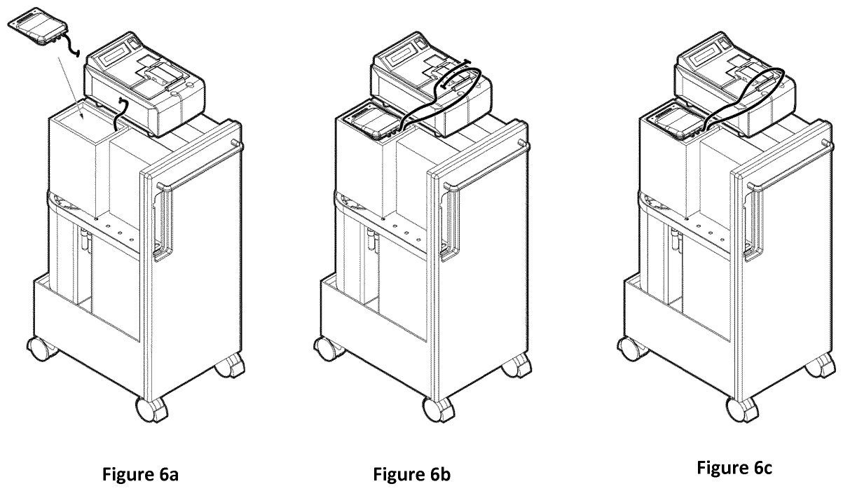

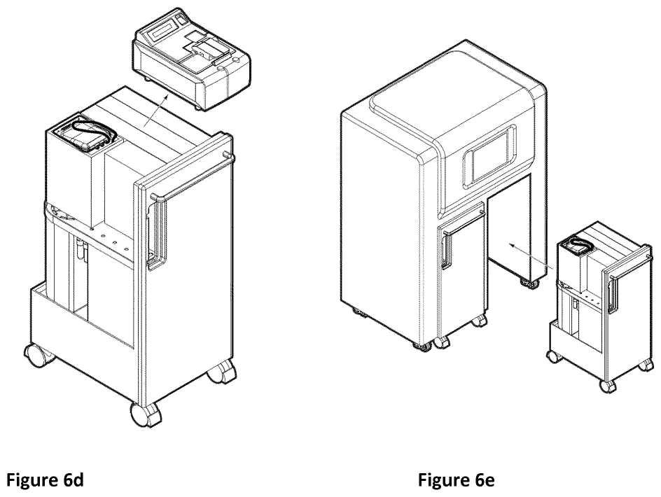

For a non-limiting example of how patient-derived material can be connected to and included in a CPM that already comprises a RP and FC, see FIGS. 6 a-e which depicts the following steps:
Step 1: Sit a tube welder on the cell processing module and load apheresis into an allocated container (FIG. 6a);
Step 2: Load an input tube from the cell processing module and an output tube form the apheresis bag into the tube welder and weld them (FIG. 6b);
Step 3: Release welded tube from the tube welder and dispose of tube ends (FIG. 6c);
Step 4: Remove the tube welder from the top of the cell processing module and place the welded tube connection on top of the apheresis bag (FIG. 6d);
Step 5: The CPM and patient cell product may now be loaded into the CPS for processing (FIG. 6e).

The RP also contains Fluidic Connection Points to enable connection between the FC and the RP to allow the two to be manufactured, supplied and loaded as separate items (see FC for more details regarding the connection methods).

It will also be noted that the RP could be integrated with the FC with chambers inside the structure prefilled with the aforementioned fluids.

The construction and layout of the RP comprises a rigid structure that houses one or more reagents vessels and fluidic connection points to connect with the FC. The rigid structure could consist of a moulded and welded polymer assembly with specific regions for discrete reagent vessels to be inserted. The individual reagent vessels could be a flexible and pliable polymer film construction with a moulded aseptic connector welded in.

The RP could also comprise of a rigid moulded polymer assembly with elastomeric over-moulded regions with connector hardware insert moulded, whereby the moulded cavities in the rigid form the vessels in which the fluid material is contained.

The reagents pack could alternatively also comprise of a series of discrete reagent vessels with integrated fluid connection points that are loaded individually. According to some specific embodiments, an empty vessel is foreseen in the reagent pack for waste disposal.

The benefits of a rigid reagents structure housing one or more individually packaged reagent fluid packs is: allowing multiple reagent manufacturing suppliers and sites; configurability of the reagents pack according to the treatment protocol; contamination prevention with multiple filling steps; along with other benefits.

By way of non-limiting example: according to particular embodiments, a configurable reagent pack may entail that only part of the reagent pack is incorporated at the start of the cellular process, while another part of the reagent pack is connected to or incorporated in the cell processing module while the process is ongoing. Of course, the connection needs to be done in a sterile fashion, without compromising the closed nature of the device or process. An example where this may be used is e.g. the provision of viral vector, which can be done just prior to a transduction step.

In an embodiment, the RP construction provides for the addition of further reagents to the RP mid process without fully removing the RP from the CPM or decoupling the fluidic connection to the FC. In an embodiment, such a construction would consist of a latchable hatch with a suitable sterile barrier that opens to expose a compartment within the RP structure whereby additional agents can be inserted and fluidically connected to the FC.

### Methods

The device and methods according to the present invention may be used to produce a wide variety of cells for use in treatment, in particular for use in cell therapy. Cell therapy is defined as the administration of live whole cells or maturation of a specific cell population in a patient for the treatment of a disease. These include, without limitation, blood transfusions, bone marrow transplantation, stem cell therapy (including hematopoietic stem cell (HSC) therapy or induced pluripotent stem cell (iPSC) therapy) and cord blood therapy. Recently, cell-based immunotherapy, wherein cells involved in the immune system are modified and administered to patients to improve the immune response to e.g. cancer or infectious diseases, has received a lot of attention. Such cell-based immunotherapies include for instance dendritic cell-based immunotherapies, NK cell immunotherapies, B cell immunotherapies, T cell immunotherapies—including, but not limited to TIL (tumor-infiltrating lymphocyte)-based therapies, TCR therapies (wherein T cells are equipped with a modified TCR) or chimeric antigen receptor (CAR) T cell therapies. Although the cell processing device described herein can be used for processing of cells to be used in any of these methods, it is particularly suited for those methods that involve modification of cells (on top of culturing or expansion of cells). This because such methods require more than one different action to be performed on the sample. In other words, the device and methods are particularly suited for cell-based immunotherapies.

Cell-based immunotherapies can be autologous (using the patient's own cells for cellular processing, after which they are reinfused or reinjected) or allogeneic (using donor cells for cellular processing, that are then administered to a patient). The device and methods can be used for both types of therapies, but the time advantage gained is most important for autologous immunotherapies, which are particularly envisaged. This is because these therapies start from patient material—when starting from donor material, the cell preparations can be prepared beforehand.

Methods that are envisaged will typically be performed in a fluidic cartridge as described herein. As this cartridge, when it is in operation, is in fluid communication with the reagent pack and provides a closed system for cell processing, it can also be said that the methods are performed in a cartridge connected to a suitable reagent pack (i.e., in a cell processing unit as described herein). Further, as the cartridge and reagent pack typically form part of a cell processing module, the methods are typically performed in a cell processing module as described herein. These cell modules are provided for use in a cell processing station, and thus the methods are particularly suitable to be performed in a cell processing device as described herein.

An advantage of the fact that the devices provide a fully closed environment with automated processing for the cellular samples is that the methods, in contrast to normal cell manufacturing methods, can be performed in non-classified areas.

The methods as described herein will typically comprise as a first step the provision of a cellular sample. The sample may be taken from a healthy subject, or from a patient in need of treatment (typically the case in autologous methods). The starting material will contain suitable cells. For cell-based immunotherapy methods, the cellular sample will typically contain cells of the immune system. Particularly, the cellular sample will contain peripheral blood mononuclear cells (PBMCs). Even more particularly, the cellular sample will contain lymphocytes. Most particularly, the cellular sample will contain T cells.

Non-limiting examples of cellular samples include blood, apheresis or leukapheresis product, bone marrow, or lymph. Blood and apheresis are particularly envisaged, as they are least invasive to collect and easily contain a sufficient number of suitable cells.

The cellular samples can then be placed in a cell processing module as described herein. This can be done by incorporating them in a reagent pack (i.e., the reagent pack will contain several vessels, one with patient-derived material, at least one with non-patient derived material), or by connecting them in a sterile way to the fluidic cartridge, or by placing them in the fluidic cartridge. It is also possible that a cell processing module is already assembled, comprising a fluidic cartridge in connection with a reagent pack (i.e. a cell processing unit), and that the cellular sample is loaded in the cell processing module (e.g. by connecting it in a sterile way to the fluidic cartridge or the reagent pack). Thus, the order in which the cellular sample, fluidic cartridge and reagent pack are provided and connected to each other are typically interchangeable and may depend on the device layout or the nature of the process to be performed. Once assembled and comprising the cellular sample, the cell processing module can then be engaged with an available receiving point on a cell processing station, and the cell processing station can be operated to perform the automated cellular processing of the cellular sample. In some embodiments, the patient material may be loaded in a cell processing module that is already engaged with a cell processing station. However, the cell processing methods will only start when the cellular sample is in (or connected to) the cell processing module. Note that, in some particular embodiments, cellular processing methods can be initiated when only part of the reagent pack is provided, and the rest of the reagent pack is provided at a later time point. This can, for example, be the case for material that is most optimally stored in a particular way, or that is normally handled in a classified environment.

A particular advantage of the methods performed in the devices as described herein is that two or more cellular processing methods can be performed on different samples on the same device, independent of each other, but essentially concurrent (i.e., at least partially overlapping in time). Thus, a method on a new cellular sample can be initiated on the same device (in a different cell processing module) prior to the processing method of the previous sample being finished. In other words, the steps of providing a cellular sample, providing a fluidic cartridge and a reagent pack, connecting the cellular sample to the fluidic cartridge (or loading the cellular sample in a cell processing module) and engaging the cell processing module can be repeated in a way that is not sequential, but concurrent (or partly in parallel). The number of concurrent processes is typically determined by the number of cell processing modules that can be engaged simultaneously in the cell processing station.

The methods can be performed on the same device independent of each other. Although this may imply that different cellular processes are concurrently performed on different cellular samples in the same device, typically it is the same cellular process that will be performed concurrently.

According to a particular embodiment, the device concurrently and independently performs the same cellular processes on different cellular samples of the same patient. This way, multiple doses for the same patient can be made on the same device.

One aspect that is particularly envisaged is that the methods of automated cell processing are used for manufacture of cell-based immunotherapies. This typically entails modification of stem cells, PBMCs, lymphocytes, NK cells, B cells, or T cells. According to some embodiments, modification is genetic modification. According to further embodiments, modification is genetic modification of T cells, e.g. by making TCR-modified T cells or CAR-modified T cells.

A typical procedure for the preparation of CAR-T cells is provided in Example 3 as an example of a process that may be suitably performed on the device according to the present invention.

Automated cell processing methods as described herein will typically comprise one or more steps selected from:
Selection and/or enrichment of the cells of interest
Activation of cells
genetic modification of cells, e.g. by transduction
Expansion of the cells
Concentration of the cells
Formulation of the cells, wherein the formulation is for preservation (including cryopreservation) and/or direct injection

In between these steps, cells will typically be washed with appropriate buffer to remove the excess reagents or in order to change media. These different steps are described in more detail below.

Selection and/or Enrichment of Cells

Typically, the cellular sample starting material will not be a pure population of the cells to be processed. Rather, patient material, such as whole blood, or partly processed patient material, such as apheresis products, are used as starting materials. These materials contain multiple cell types, and to select for and/or enrich the particular cell type to be processed, different methods can be used (depending also on the cell type). As many of the cell types used in cell-based immunotherapy are PBMCs of one sort or another, enrichment of cells can also be done by enriching or selecting for PBMCs (thereby enriching more than one type of cells), which may or may not be followed with further enrichment or selection for the specific cell type (e.g. T cells, NK cells, B cells, monocytes, lymphocytes).

PBMC selection on a sample most typically is performed using a Ficoll procedure using a simple and rapid centrifugation procedure based on the methods originally developed by Boyum and well-known in the art (Isolation of mononuclear cells and granulocytes from human blood. (Paper IV). Boyum, A., Scand. J., Clin. Lab. Invest. 21 Suppl, 97, 77-89 (1968); Isolation of leucocytes from human blood—further observations. (Paper II). Boyum, A., Scand. J. Clin. Lab. Invest. 21 Suppl, 97, 31-50 (1968); Isolation of lymphocytes, granulocytes and macrophages. Boyum, A., Scand. J., Immunol. 5 Suppl, 5, 9-15 (1976)).

In the methods provided herein, depending on the design of the cell processing device or the fluidic cartridge, enrichment or selection of PBMCs (or, equivalently, separation of PBMCs of non-PBMCs) can also be achieved by:
Freezing the cells, which kills red blood cell (RBCs) and granulocytes, and allows T-cells and monocytes to survive;
Using micro fluidic channels, the RBCs can be removed from the blood sample (typically based on their different phenotype: the different size, weight, volume, light scattering or markers can be used to guide the different cells in a microfluidic circuit);
By using a hydrocyclone, or several stages of hydrocyclones, the RBCs can be removed from the blood sample because of their different ratio of centripetal force to fluid resistance;
Using multiple layers of filter sizes, the T-cells can be positively selected from the sample, removing RBCs and granulocytes. Additional filters can be used to perform the other wash steps;
Using magnetic beads/clouds, the PMBCs can be isolated using negative selection to remove RBCs/granulocytes (lysing and mag beads), and positive selection of the T-cells and monocytes;
Using the different densities of RBC and PBMCs, a centrifuge could be used to collect only the PBMCs from whole blood;
By using a counter-flow centrifuge (CFC), we can separate out the RBC and granulocytes from the T-cells and perform the several wash steps;
Using fibre filtration, a media exchange can take place to concentrate the cells during the wash stages. Tangential flow hollow fibre filters are useful for this;
Red blood cells can be lysed using a RBC lysis buffer such as an ammonium chloride (NH₄Cl) solution;
Hetastarch can be used as a sedimenting agent for RBC;
Magcloudz™ gel coated with streptavidin used in combination with biotin-conjugated CD3 antibodies can be used to positively select T cells;
Using acoustic technology to generate multi-dimensional standing waves to separate RBC cells.

Also, positive or negative selection steps for specific cells (typically using markers) can be used in the methods to achieve selection or enrichment of cells. For instance, CD3, CD4 and CD8 markers can be used to enrich T cells, CD19 and CD20 for human B cells, CD11c, CD123, BDCA-2, BDCA-4 for dendritic cells, CD56 for human NK cells, CD34 for hematopoietic stem cells, CD14 and CD33 for human monocytes or macrophages, CD66b for human granulocytes, CD41, CD61, CD62 for human platelets, CD235a for erythrocytes. The latter three cell types will typically be negatively selected (i.e., removed from the sample). Antibodies against these markers are well known, commercially available and routinely used in selection or enrichment of these cell types. Antibodies can be used in these methods using surfaces coated with these antibodies (e.g. in the fluidic cartridge), using columns with such antibodies, using (para)magnetic beads with these antibodies (for ease of manipulation), using (para)magnetic beads that can be coupled to these antibodies (e.g. using MagCloudz™ kits; Quad Technologies®) or the like.

### Activation of Cells

Once an appropriate cell population has been achieved (either directly as a cellular sample, or after a step of selecting and/or enriching the appropriate cells), the methods may further contain a step of activating the cells. This step is optional in most cases, but is particularly useful when the methods entail modification of the cells using (gamma-)retrovirus or a (gamma-)retroviral vector. This because retrovirus (depending on the strain) preferentially or exclusively transduces dividing cells. Lentiviruses and adenoviruses (and the vectors based thereon) typically also infect both dividing and non-dividing cells, so activation of cells is not a prerequisite. The same goes for non-viral methods (e.g. electroporation, sonoporation, transfection, magnetofection, gene gun).

Activation of cells is done as described in the art. Typically, CD3 antibody (OKT3) is used as an activation signal. A second activation signal can also be used, e.g. an anti-CD28 antibody. Such second signal of activation can also be used as the sole activation signal, although anti-CD3 based activation is the most common in the art. Other activation signals are e.g. anti-CD2 antibody, anti-4-1BB antibody. Cytokines that can be used to activate cells include, but are not limited to, IL-2, IL-7, IL-12, IL-15, and IL-21, either alone or in combination.

### Genetic Modification of Cells

The methods described herein will often involve a step of genetic modification of cells. Genetic modification can be done using standard procedures in the art, including, but not limited to, transfection, electroporation, electrofusion, sonoporation, magnetofection or a gene gun. According to particular embodiments, genetic modification is done through transduction with viral vectors. Viral vectors include, but are not limited to, retroviral vectors, lentiviral vectors, and adenoviral vectors.

Genetic modification protocols are well known in the art and are compatible with the methods described herein, and can be accommodated in the cell processing devices described herein (provided that the appropriate hardware is integrated in the device, the cell processing module, or the fluidic cartridge). The efficiency of some viral transduction protocols (particularly retrovirus, but also lentivirus) is enhanced when cationic agents are used, such as e.g. RetroNectin® (a fibronectin fragment), polybrene (hexadimethrine bromide), or vectofusin-1. Thus, the methods of genetic modification may entail the use of these reagents, e.g. using RetroNectin® coated plates or bags. Other exemplary ways the genetic modification step may be implemented in the methods is through the use of
Magcloudz™ coated with RetroNectin® (or similar product) and suspended in a CFC cone;
Transduction can be achieved with the cone of a CFC being coated with RetroNectin® (or similar product);
Transduction can be achieved with the cone of a CFC creating a fluidize bed where cells and viral vector interact;
Transduction can be achieved by introducing the cells and viral vector into a filter that has been primed with a suitable primer such as RetroNectin®, or without the priming step;
Transduction can be achieved by concentrating cells with viral vector using acoustic technologies.

The type of genetic modification (i.e., which construct is introduced or which protein's expression is altered) is not limiting the invention: any genetic modification can be made. Particularly envisaged for cellular immunotherapy however is the creation of TCR-modified T cells (by introduction of a modified TCR through genetic modification) or CAR-modified T cells, NK cells or the like.

Examples of CAR constructs that can be introduced in genetic modification protocols include, but are not limited to, CARs directed against the following targets: CD19, BCMA, CD20, CD22, CD30, CD33, CD38, CD70, CD123, CEA, c-Met, CS1, EGFRvIII, EpCAM, ERRB2, HER-2/neu, folate receptor alpha, GD2, IL-13Ra2, L1-CAM, mesothelin, MUC1, MUC16, PSCA, PSMA, TAG-72, NKG2D, NKp30, and B7H6. CARs may be first generation constructs (with only one stimulatory domain, typically CD3 or FcεRI), second generation constructs (with an additional stimulatory domain, typically CD28 or 4-1BB), or third generation constructs (with two additional stimulatory domains, typically one selected of the group already mentioned and OX40, ICOS, DAP10, DAP12, CD2, CD27, CD30, CD40 or similar chains). They may also be further modified (inhibitory CARs, gated CARs, sideCARs, TRUCKs, armored CARs and the like). Also specifically envisaged is the use of bispecific CARs, with specificity for two different targets.

Particularly envisaged is the use of NKG2D CARs (as described in WO2006036445), B7H6 CARs (as described in WO2013169691) or NKp30 CARs (as described in WO2013033626).

Modified TCRs have the advantage over CARs that they can also target intracellular proteins. In addition to the targets envisaged by the CARs, examples of targets envisaged by modified TCRs include, but are not limited to: NY-ESO-1, MAGE-A3, ROR-1, WT-1.

### Expansion

At different stages of the process, the cells may be cultured or expanded, to increase the number of processed cells obtained at the end of the processing methods. For instance, cells may be expanded before or after activation, or after modification, or prior to concentration or formulation. Typically, expansion of cells can be done in a gas permeable rapid expansion device, such as the G-REX™ system (Wilson Wolf®).

However, the current methods are not limited to the use of G-REX™; the following alternatives are also envisaged, and a skilled person can find similar ways of expanding cells.
Hollow fiber: Cells are loaded into the centre of the hollow fibres of the filter tubes for activation and/or expansion. Growth media and cytokines can be circulated around the outside of the fibres and therefore feed/contact the cells through the fibre;
Custom Flask: the bottom of the flask would be constructed of similar material to the G-Rex flask for gas permeability;
Cell culture bags without rocking;
Cell culture bags with rocking, such as for instance in the single use WAVE Bioreactor™ (GE Healthcare®);
Hydrogel with antibodies attached: this forms an effective, large surface area for cells to be activated and expand. If required additional gel could be added. The hydrogel could be contained in a flask, bag or CFC container.
Microcarrier beads can be used in any application where anchorage-dependent cells are to be cultured.

Note that the surfaces used for expansion of cells may also be used in vessels for other process steps (e.g. an activation step could also occur using hollow fibres or microcarrier beads).

### Washing, Concentration, Formulation

The methods may contain one or more steps of washing the cells, and/or of concentrating the cells. These can be envisaged according to known procedures. By way of non-limiting, Example 3 specifies several of these steps.

Typical buffers envisaged for washing of cells include, but are not limited to, phosphate-buffered saline (PBS) or Hanks' Balanced Salt Solution (HBSS).

Steps for concentration typically are performed to reduce the volume, and entail the selective removal of fluids, while retaining the cells in the sample. Although concentration steps can be performed throughout the process, most typically, a concentration step is envisaged at the end of the process, prior to formulation. Suitable technologies that can be used for concentrating the cells (or reducing the volume of the sample include, but are not limited to, counterflow centrifuge (CFC), hollow fibre filter (HFF), tangential flow filtration (TFF), acoustic technology.

The end results of the methods will typically be an expanded number of cells that are suitably formulated. If formulation is for direct administration to the patient (typically direct injection or direct infusion), the fresh product will typically be formulated in a suitable medium that contains a saline buffer and serum, as detailed in the art. “Direct administration” as used herein refers to the fact that the product is typically administered within 24 hours (i.e., no long-term storage or addition of stabilizing agents is required) and the product is not frozen prior to administration. It does not necessarily imply that the product is immediately taken from the cell processing device to the patient.

When the formulation of cells is for preservation, typically preservation solutions will be used or added to the formulation, particularly for hypothermic preservation. When formulation is done for cryopreservation, typically cryoprotectants will be added to the cells, such as DMSO or glycerol. Formulations for cryopreservation are well known in the art, and may also contain trehalose, dextran, ethylene glycol, propylene glycol or the like.

### QC Testing

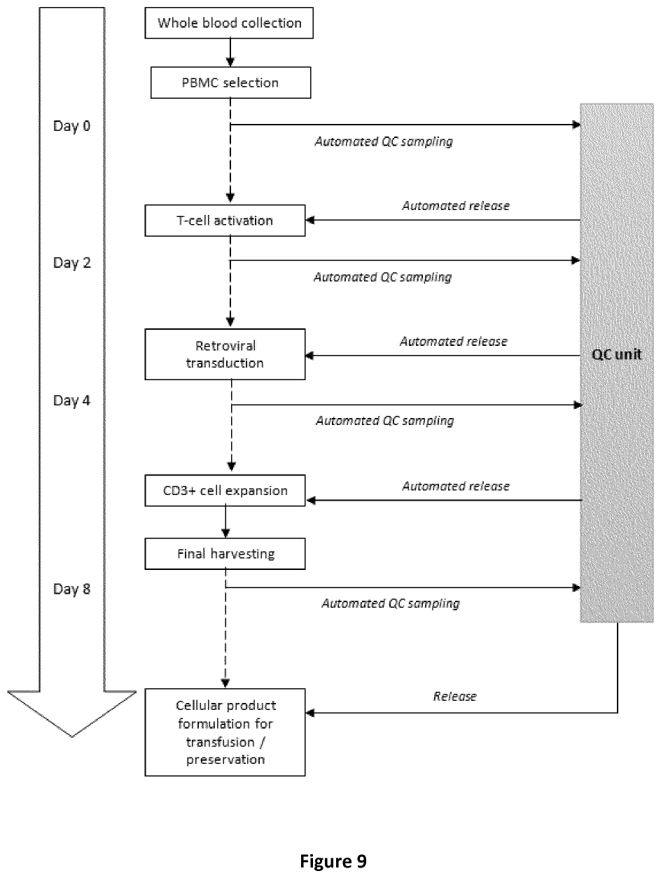

In addition to all the method steps for manufacturing of the processed cells (from providing the cellular sample over modification of the cells to formulating the cells for preservation or direct administration), the methods may further comprise steps for quality control at different stages of the process. As with the other steps, the QC sampling and testing is done fully automated and in the closed system. A schematic overview of different QC sampling steps for an exemplary cell processing method (here, a method of retroviral transduction of T cells) is provided in FIG. 9.

QC method steps that can be incorporated in the cell processing methods described herein may e.g. comprise the monitoring of cell counting and viability by microscopy, fluorescence, trypan blue, or flow cytometry; the monitoring of cell identity, purity, or homogeneity by flow cytometry or other methods; the detection of endotoxins (such as by a LAL test); monitoring of safety (e.g. by PCR of Viral Copy Number (VCN) and/or detection of Replication Competent Retrovirus (RCR-test)); Mycoplasma detection (e.g. by PCR or Gram staining); monitoring of potency (e.g. by co-culture with cell lines).

The present invention addresses several problems. Firstly, in the system(s) described herein, the high capital cost of a machine for manufacturing cell therapy is addressed by the unification of CPMs to reduce system cost with the benefit of allowing sharing and reuse of costly components within the CPS.

The sharing of common facilities by multiple CPMs in the CPS also has a benefit in reducing the machine footprint required to process multiple patient samples. Floor space in clean rooms tends to be limited due to the expense in acquiring, commissioning and operating such space, and therefore a reduction on the need for floor space in this environment leads to a reduction in facility costs.

In addition, the provision of a closed sterile system allows the cell manufacture to be performed in an unclassified environment rather than in a designated cleanroom. Thus, not only is the device more compact (and thus demanding less space) by incorporating different cell processing steps in one device, it also has fewer demands on the nature of the space. With the device of the present invention, there is no or minimal requirement for controlled aseptic conditions such as laminar flow cupboards and personal protective equipment. This eliminates the need for and maintenance of these expensive resources. This also reduces or eliminates the need for enhanced training of operators who would otherwise be required to be familiar with the techniques and equipment required for working under aseptic conditions.

Reduction in size and cost of the equipment, and the reduction or removal of the need for clean room space makes it easier the system of the present invention to be located near to the patient, in a hospital, for example. This proximity of the patient and the manufacturing location reduces the likelihood of error due to traceability of patient material to the manufacturing location. Traceability management can be achieved more easily in a single location (for example using IDs on the disposable cartridge that are matched to IDs on wrist bands for the patient, so that traceability is maintained from collecting samples to until after the therapy is returned to the patient). This reduces or eliminates the risk associated with information changing hands that is typically associated with failures of logistics in conventional systems.

Further advantages of close proximity of the patient and the location for sample processing can be summarised as follows:
the reduction of the onerous logistics, including temperature management, of patient material during transport to the manufacturing location;
the reduction of the cost of transport of patient material to the manufacturing location. Transport of patient material can be implemented in the patient material collection Standard Operating Procedure (SOP). This means the cost of a third party arranging the transport is eliminated. This also avoids an additional potential source of error/mislabeling.

A further problem addressed by the system of the present invention is reduction or avoidance of inadvertent destruction or loss of patient material due to operator error in manual processes. The manufacturing process within the device is automated to eliminate the need for operator interventions and complex, dexterous manual manipulations. Operator interaction is reduced to a minimum throughout the entire automation of the process thereby reducing instances of operator error.

Likewise, optimisation of system processes can be achieved through in situ real-time measurements (such as cell counting) and testing (such as potency) that are performed by the automated system. Results of such analysis can be fed directly into pending manufacturing decisions for that sample (i.e. leave for longer in expansion). This has the benefit of operator interaction being reduced to a minimum throughout the entire automation of the process thereby reducing instances of operator error.

Loss of patient material due to contamination caused by open processing in traditional laboratory conditions is addressed by the present invention by providing a ‘functionally closed’ sterile or aseptic processing environment within the FC. that may be integrity tested by the automated system. This way, the contamination risk is essentially eliminated.

Furthermore, integration of automated quality control and product analysis addresses the disadvantages of the high cost of using laboratory based analysis and the potential for operator error resulting from manual handling of samples.

Costs due to the requirement for specialized operators to perform complex manual tasks, including cost of salaries, cost of training and cost of validating resource training, is also addressed by an optimized automated system being responsible for the complex tasks of the process. This automation approach negates the need for skilled operators and their associated training costs. Ideally, the CPS is designed for maximal user-friendliness, and the messages displayed on the user interface display will not require specialized operators for understanding. The need for specialized operators at the location of the device can be further reduced by sending potential error messages to the manufacturer or supplier of the device, so that the manufacturer or supplier can provide assistance (rather than e.g. the hospital employing technical experts).

Finally, the system of the present invention provides a significant advantage in the flexibility of providing independent parallel processing of patient samples. Each CPM may be prepared and loaded into the CPS independently as the need exists. This may be done without consideration of the state of operation of the system or any samples that have previously begun processing in neighbouring CPMs in the same machine. The system will simply allocate resources to each CPM as it needs it. As it is envisaged that the majority if not all of the cellular processing will be conducted within the individual FC within each CPM then the risk of delay caused due to waiting for shared resources is reduced and machine run time efficiency is maintained at a high level. This has significant benefits in environments where the throughput of cell processing is irregular, for example, in a clinical environment where patient samples are removed based on the timing of patient need, and yet an efficient, fast turnaround of cell processing is required to optimise patient care. Efficient processing of samples, minimal space requirements, and the other cost advantages outlined above carries an overall advantage in lower cost of goods for the therapy.

### EXAMPLES

### Example 1: Cell Processing System According to the Present Invention

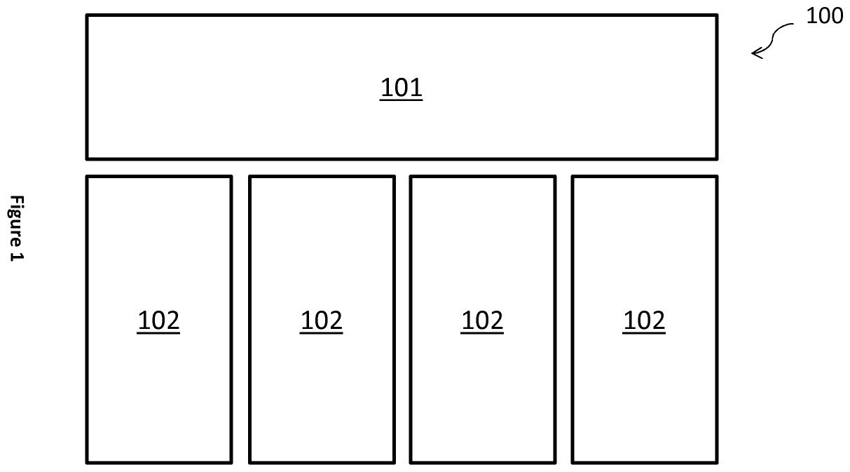

As best shown in FIG. 1, an embodiment of the cell processing device 100 of the present invention comprises a cell processing station 101 with which one or more cell processing modules 102 integrates. In FIG. 2a, one embodiment of a cell processing device 100 is shown with the cell processing module 102 mounted on a trolley so that it may be wheeled around the manufacturing facility to collect reagents and other items required for the process, or to collect samples from the patient. In this embodiment, the fluidic cartridge 103 is estimated to weigh 15 kg and the reagent pack weight is estimated at 40 kg.

Mounting the cell processing module on a trolley provides a solution to manual handling required for the collection and loading of disposable components that are either too large or too heavy to be safely or comfortably carried by hand. The trolley is deigned to interface accurately and specifically with the various components of the cell processing module 102 allowing them to be pre-connected together before they are loaded into the cell processing station 101. In this way, the operator can collect the various components required for the process from their respective storage facilities and while loading them on to the trolley connecting them together at the same time. Once all components are loaded onto the trolley the operator would direct the trolley into the trolley dock where the cell processing station would interface with the trolley and cell processing module.

The trolley has a rigid construction fabricated from a metal structure fitted with locating features that hold the cell processing module 102 in a precise location such that when the trolley is docked with the cell processing station 101, alignment with the cell processing module 102 with the cell processing station interface is correct. Docking the trolley with the cell processing station 101 is achieved initially by approximate guidance using bumpers (not shown). Once the trolley is engaged there are linear guides (not shown) that engage with the cell processing module 102; these guides are ultimately responsible for accurate alignment of the cell processing module 102 to the cell processing station 101. This alignment is made possible by the disposable mass being supported on an articulated balance allowing the cell processing module 102 to be lifted or lowered to achieve final alignment without adding resistance to the operator during docking. The articulation and mass balance is inactive prior to docking of the trolley to ensure the cell processing module 102 is transported in a secure manner. Once the front of the trolley engages with the cell processing station 101 the mass balance lock is released and the cell processing module 102 can be finally located by the alignment rails.

The cell processing station 101 is shown with a centralised user interface 105.

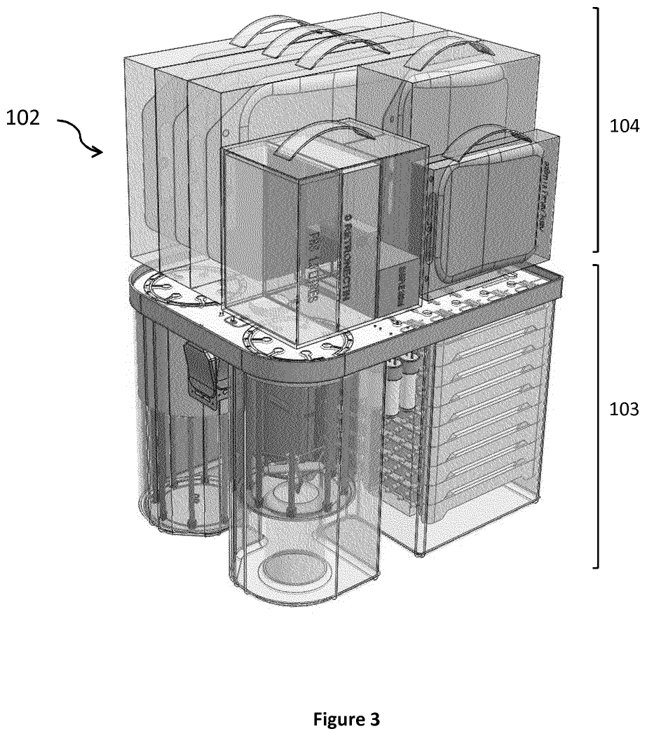

The cell processing module 102 comprises a fluidic cartridge 103 and a reagent pack 104. A typical cell processing module is shown in assembled form in FIG. 3. The reagent pack 104 is located on top of the fluidic cartridge 103. When the reagents are installed, there is a sterile fluid connection established between the reagent pack 104 and the fluidic cartridge 103.

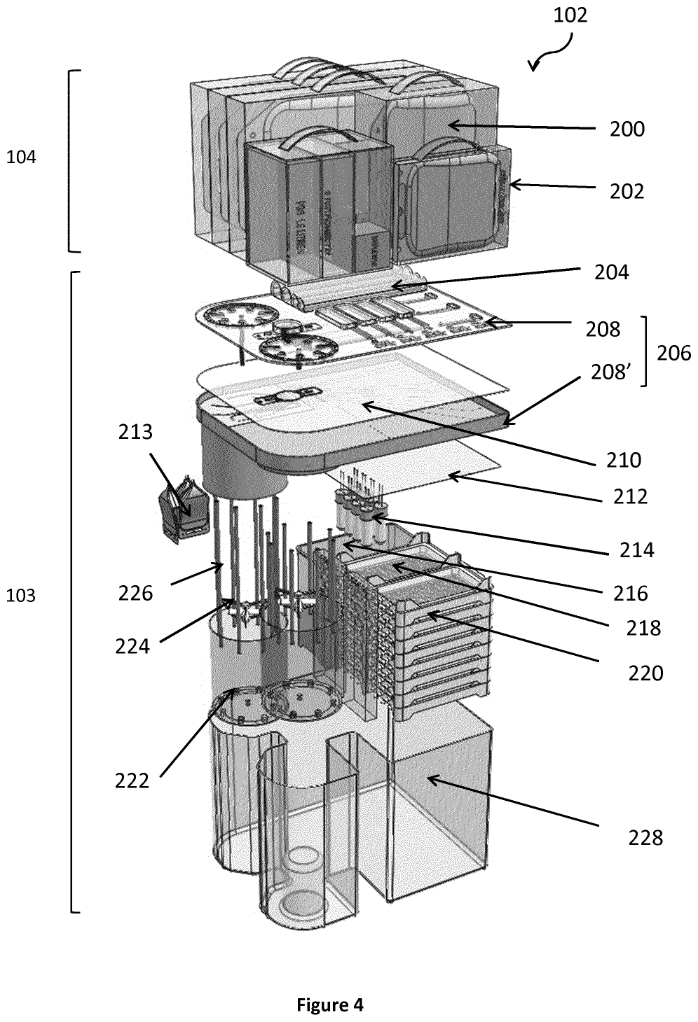

FIG. 4 shows the cell processing module 102 in exploded form. Bulk reagents are stored in bags 200 within a number of boxes 202 that together form the reagent pack 104. The boxes 202 function as an outer shell and are loaded with an empty or pre-filled bag where the box is then closed as part of the manufacturing process by some mechanical means where the operator of the system application cannot easily open it without the use of abnormal force or tools. The boxes 202 also provide a means of locating and supporting the connection point for the fluid inlet and or outlet to the bag 200. Another function of the box 202 is to provide a means of location, this can be for the connection process of the RP to the FC or simply to allow the RPs to stack for bulk storage and transport. The construction of the box can be either a folded plastic construction or fully moulded with a secondary or hinged lid. The box must not provide an air tight seal as this will prevent the fluid from being drawn out of the bag. The reagent pack 104 being divided into separate units accommodates storage of reagents, and promotes ease of manual handling.

Hollow fibre filters 204 are encased in a fluidic circuit layer 206. The fluidic circuit layer 206 is comprised of two moulded forms 208, 208′ sealed with a silicon membrane 210 and polycarbonate (PC) capping film 212 on the bottom. Output (cryo) bags 213 are part of the fluidic circuit layer 206 and removed at the end of the process. QC and IPC samples 214 are stored during processing and are externally accessible if off-line testing is required.

An expansion chamber 216 comprising three silicon membrane based vessels is shown, along with transduction vessels 218, activation vessels 220, and mixing vessels 222 containing stirring impellers 224 and multiple syphon tubes 226. The syphon tubes 226 are used to add and remove material and also to rinse the chamber for better cell recovery.

A lower tub 228 forms the outer shell for the fluidic cartridge 103.

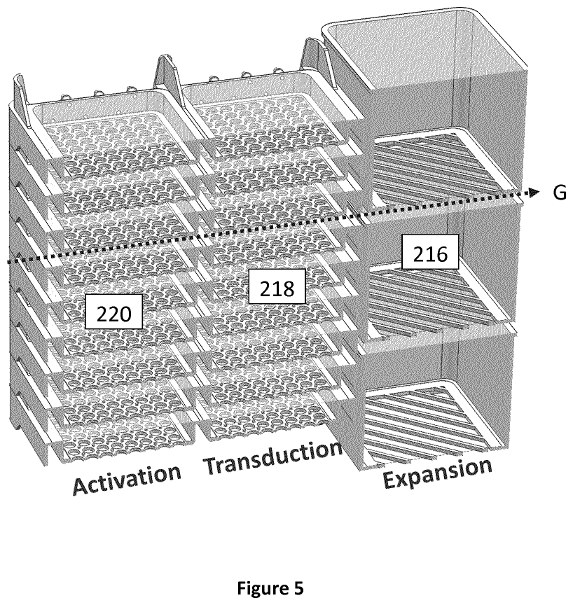

FIG. 5 shows the culture vessel layout of the fluidic cartridge 103 which comprises the expansion chamber 216. transduction vessels 218, and activation vessels 220. The construction of the activation vessels 220 and the transduction vessels 218 uses a frame with FEP film bonded top and bottom. Vessels 220, 218 are stacked to achieve the required area dependent on the process and provide design flexibility should this need to be increased or decreased. The construction of the expansion chamber 216 uses silicon film bonded top and bottom. The design allows gas G to pass between levels allowing good exchange across membranes.

### Example 2: Detailed Description of Process Workflow in Apparatus According to the Present Invention

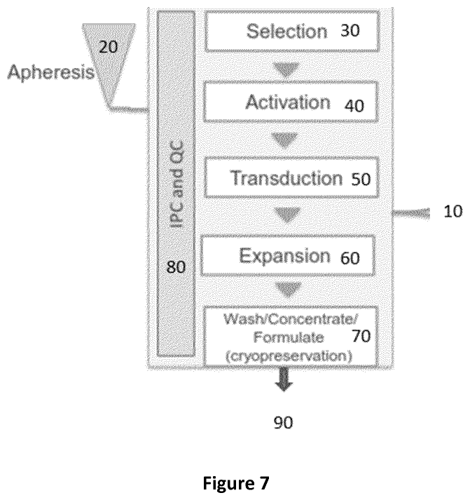

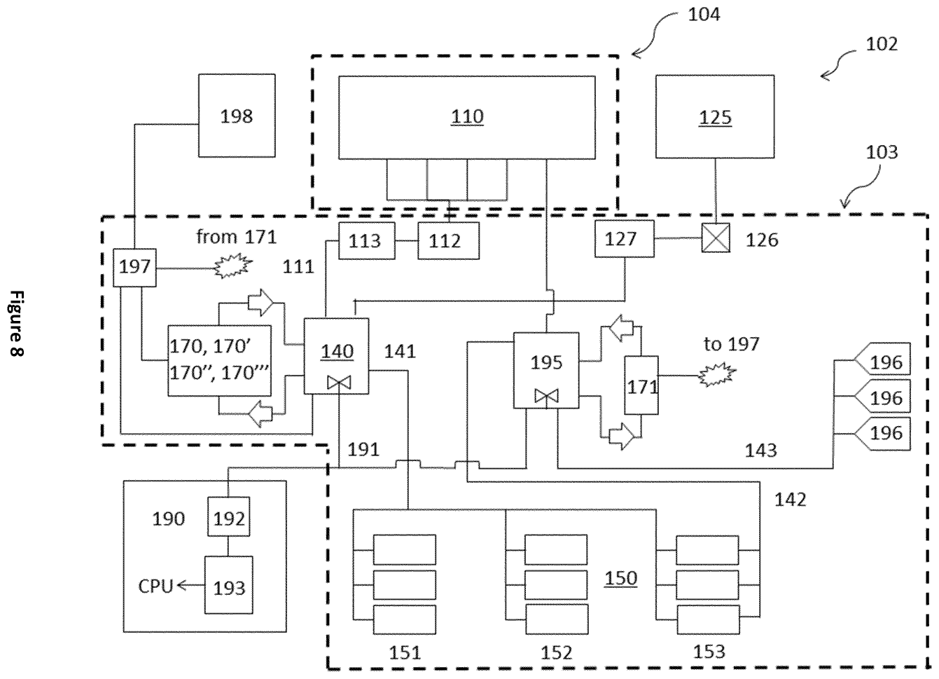

In a specific embodiment of the invention there is provided a cell processing device 100 that operates according to the process flow shown in FIGS. 7 and 8.

A sample 20 comprising cellular material obtained from a human or animal subject is obtained. The sample may be obtained by apheresis techniques that are known in the art. The sample 20 is introduced into the workflow 10 which comprises a series of process steps that enable the selection and isolation of particular cell types comprised within the sample 20, as well as the appropriate processing of that material to produce the desired output cellular product 90.

In one embodiment apheresis cellular sample material 20 is introduced into the workflow 10. The material progresses through a selection step 30 where cellular material is fractionated or otherwise processed in order to identify the desired sub-type of cells—for example, T cells. The isolated sub-type of cellular material progresses to an optional activation step 40 where the desired cellular sub-type can be activated, if appropriate to that cell type. The cellular material is subjected to a transduction step 50 in which exposure to one or more growth factors and/or transfection of the cellular material with one or more recombinant vectors may occur. The resultant cellular material is progressed to an expansion step 60 allowing for passaging of the cells and facilitating expansion of the desired processed cellular material. Upon reaching a predetermined threshold benchmark for desired expansion, the cells are transferred to the formulation step 70 where they are processed to provide a final cellular product 90. Formulation 70 may comprise concentration of cellular material, combination with preservation solution as well as cryopreservation where required. Throughout the process 10 quality control and monitoring of process parameters is undertaken by analytics steps 80 that are integrated with and capable of exercising feedback control on each of the aforementioned process steps.

In one embodiment of the invention the process 10 is performed within a cell processing device 100. The device 100 comprises a cell processing module 101 that includes a reagent pack 104 that includes and incorporates multiple reagents, and a fluidic cartridge 103 that incorporates liquid handling and processing apparatus that together contribute to the formation of a system that is suitable for performance of the process 10. Hence, the cell processing module 101, once assembled, defines a portable system that is closed to the external environment, save for input and output ports, which allow for cellular processing to be conducted with minimal risk of contamination and with high levels of integrity of identity of the cellular material throughout the procedure.

Within the cell processing module 101 cellular material derived from a subject, suitably a patient, is introduced to the fluidic cartridge 103 via an input vessel 125. Typically the input vessel 125 is configured to hold the products of an apheresis procedure and will include a bag or pouch comprising a suitable biocompatible polymer material. The volume of the input vessel 125 may vary but will likely range from at least 50 ml to at most 3000 ml, although for most apheresis procedures the volume will typically very between 100 and 300 ml, and the vessel 125 will then accordingly accommodate this range of volume of apheresis cellular material in a liquid or gel state. The input vessel 125 engages with the fluidic cartridge 103 via a fluidic connection made using a sterile tube welder where the tube from vessel 125 can be joined to the input tube protruding from the fluidic cartridge 103. An alternative means of connection may be made using a mechanical sterile fluidic connector, vessel 125 may be received from a third-party tube set and therefore require the mating connector to be fitted before it is connected to the fluidic cartridge 103. Cellular material is introduced into the fluidic cartridge 103 via actuation of a valve 126 which in turn directs flow of the cellular material into a dosing chamber 127 located within the fluidic cartridge 103. Throughout the system, fluid flow may be actuated via application of external pressure, mechanical pumps (including peristatic pumps) or capillary flow as appropriate.

Within the reagent pack 104, a plurality of reagent vessels 110 provide a range of reagents that are necessary for carrying out the process 10. The reagent vessels 110 suitably comprise appropriate growth media for the various phases of cell culture, activation and expansion; lytic agents (e.g. ammonium chloride); growth factors; nutrients; transfection reagents, including viral vectors; and preservation solution. The reagent vessels 110 are maintained in fluid communication with a processing chamber 140 located within the fluidic cartridge 103. Reagents may be introduced from any one of the reagent vessels 110 to the processing chamber 140 via separately actuated supply lines 111. As appropriate to the respective reagent, the supply line 111 may further comprise an intervening mixing chamber 112 and dosing vessel 113. In this way reagents which are kept in concentrated, lyophilised or in another storage form within the reagent vessels 110 may be suitably reconstituted to standard pre-defined concentrations or combined into acceptable dosage formulations prior to being introduced into the processing chamber 140. It is optional that the processing chamber 140 is physically comprised within a planar stage, or zone. The planar stage may comprise a variety of supplementary cell processing systems that are in fluid communication with each other and, importantly, with the processing chamber 140.

The processing chamber 140 defines sufficient volume in order to undertake adequate mixing of solutions comprising cellular material and reagents. In addition, the processing chamber 140 comprises mixing apparatus that may comprise a magnetic impeller or other rotating mechanical mixer.

Cellular material comprised within the dosing chamber 127 may be introduced into the processing chamber 140 where it is held for the initial selection step 30 of the process 10. In order to lyse unwanted red blood cells comprised within the cellular material (derived from the apheresis procedure) ammonium chloride is introduced from the reagent vessels 110 into the processing chamber 140 to achieve a concentration of approximately 0.8%. Following mixing and incubation the treated cellular material is pumped from the processing chamber 140 to the first of a series of tangential flow filtration circuits 170 located within the fluidic cartridge 103. Suitably the tangential flow filtration circuit comprises a hollow fibre filter that permits for separation of cellular retentate from unwanted excess solution volume, reagents and lysed cellular material to a waste vessel 197. At any point in the process 10 effluent waste collected within the waste vessel 197 may be voided to a sealed waste storage chamber 198. Cellular material comprised within the retentate flow is returned to the processing chamber 140 where a defined volume of media is introduced from the reagent vessels 110. The retentate-media mixture comprised within the processing chamber 140 may be recirculated once or more times through the filtration circuits 170 followed by washing with additional media to ensure removal of unwanted reagents or waste material from the selection step 30. Product of the selection step 30 is returned to the processing chamber 140.

At any point in the process 10, but typically at the end or beginning of a defined process step, a controlled volume, or aliquot, of the processed cellular material may be aspirated and transferred to the analytics module 190 via analytics supply lines 191 that maintain fluid communication between the processing chamber 140 and the analytics module 190. The controlled volume of processed cellular material passes along the supply line 191 into the analytics module 190 where it may be held within a dosing vessel 192. In process cell counting may be performed by a cell counter 193 on the volume comprised within the dosing vessel 192. The cell counter 193 is in electronic communication with a CPU comprised within the device 100 (not shown). The results of the analytics steps 80 inform decisions within the process 10 as a whole. Hence, the results of the in process cell count determine whether additional media is introduced from the reagent vessels 110 to the processing chamber 140 in order to control cell concentration. Alternatively, if there is a failure at this or any stage in the process 10 the process may be paused or the operator of the device 100 may be alerted.

A non-limiting example of a process flow for quality control and analytics in an automated process for the production of T-cells according to an embodiment of the present invention is provided in FIG. 9.

In order to commence the activation step 40 cell media may be combined with lyophilised cytokine comprised within the reagent vessels 110 in a reagent mixing chamber 112. A controlled dose of reconstituted cytokine solution is transferred via a supply line 111 to the processing chamber 140 where is it combined and mixed with the cell product of the selection step 30 to generate an activation cell formulation. A series of controlled doses of activation cell formulation are sequentially transferred via a supply line 141 to a plurality of activation surfaces 151. The activation surfaces are fabricated soft or rigid vessels that comprise of horizontal surfaces facilitating cell adherence and gas permeation. The activation surfaces 151 are one or more individual compartments that may be individually filled and emptied by the fluidic circuit. The activation surfaces 151 are comprised within a discrete compartment 150 within the fluidic cartridge 103 that is separated from the planar processing zone that comprises the processing chamber 140. Separation of the activation surfaces 151 permits independent regulation of environmental conditions for the extended periods of time that may be necessary, i.e. incubation, for the activation step 40 to be completed. Any excess activation cell formulation may be discharged to the waste vessel 197 and discarded as previously described. Following discharge of activation cell formulation from the processing chamber 140 the chamber is purged with additional media the reagent vessels 110, with resultant effluent also routed to the waste vessel 197.

Upon completion of the activation step 40 the activated cells are harvested by transferring activated cell product from each of the activation surfaces 151 back through the supply line 141 to the processing chamber 140. The combined activated cell product is mixed within the processing chamber 140 to ensure homogeneity. The activated cell product is then is pumped from the processing chamber 140 to a second tangential flow filtration circuit 170′ thereby facilitating concentration of cellular retentate and removal of unwanted spent media to the waste vessel 197. The concentrated activated cell product is returned to the processing chamber 140. A controlled volume of concentrated activated cell product is aspirated and subjected to an in process cell count within the analytics module 190, as described previously. The results of the in process cell count determine whether additional media is introduced from the reagent vessels 110 to the processing chamber 140 in order to control cell concentration.

Commencement of the transduction step 50 occurs when a controlled dose of viral vector is transferred from the reagent vessels 110 in the reagent pack 104 via a supply line 111 to the processing chamber 140 where is it combined and mixed with the cell product of the activation step 40 to generate a transduction cell formulation. A series of controlled doses of transduction cell formulation are sequentially transferred via a supply line 141 to a plurality of transduction surfaces 152. The transduction surfaces 152 are fabricated soft or rigid vessels that comprise of horizontal surfaces providing gas permeation. The transduction takes place on the lower surface where a surface optimised for cell adhesion is not required but also not excluded form this invention description. The transduction surfaces 152 are one or more individual, stackable, compartments that may be individually filled and emptied by the fluidic circuit. The transduction surfaces 152 are also comprised in the compartment 150 within the fluidic cartridge 103 that is separated from the planar processing zone. Any excess transduction cell formulation may be discharged to the waste vessel 197 and discarded as previously described. Following discharge of transduction cell formulation from the processing chamber 140 the chamber is purged with additional media the reagent vessels 110, with resultant effluent also routed to the waste vessel 197.

Upon completion of the transduction step 50 the transduced cells are harvested by transferring transduced cell product from each of the transduction surfaces 152 back through the supply line 141 to the processing chamber 140. The combined transduced cell product is mixed within the processing chamber 140 to ensure homogeneity. The transduced cell product is then is pumped from the processing chamber 140 to a third tangential flow filtration circuit 170″ thereby facilitating concentration of cellular retentate and removal of unwanted spent media to the waste vessel 197. The concentrated transduced cell product is returned to the processing chamber 140. A controlled volume of concentrated transduced cell product is aspirated and subjected to an in process cell count within the analytics module 190, as described above. The results of the in process cell count determine whether additional media is introduced from the reagent vessels 110 to the processing chamber 140 in order to control cell concentration within the concentrated transduced cell product.

Initiation of the expansion step 60 occurs when cell media is combined with lyophilised growth factors and suitable signalling/proliferation promoting molecules comprised within the reagent vessels 110 in a reagent mixing chamber 112 to generate an expansion cell media. A controlled dose of expansion cell media is transferred via a supply line 111 to the processing chamber 140 where is it combined and mixed with the cell product of the transduction step 50 to generate a cell expansion formulation. A series of controlled doses of cell expansion formulation are sequentially transferred via a supply line 141 to a plurality of expansion surfaces 153. The expansion surfaces 153 are also comprised within the discrete compartment 150 within the fluidic cartridge 103 that is separated from the planar processing zone. Excess expansion cell formulation may be discharged to the waste vessel 197 and discarded as previously described. Following discharge of expansion cell formulation from the processing chamber 140 the chamber may be purged with additional media the reagent vessels 110, with resultant effluent also directed to the waste vessel 197.

Upon completion of the expansion step 60 the expanded cells are harvested by transferring cell product from each of the expansion surfaces 153 through the formulation supply line 142 to the formulation chamber 195. The combined cell product is mixed within the formulation chamber 195 to ensure homogeneity. The activated cell product is then is pumped from the processing chamber 140 to a tangential flow filtration circuit 171 thereby facilitating concentration of the cell product formulation within a retentate and removal of unwanted spent media to the waste vessel 197. The concentrated cell product formulation is returned to the formulation chamber 195. A controlled volume of concentrated cell product formulation is aspirated and subjected to an in process cell count within the analytics module 190, as described previously. The results of the in process cell count determine quality control and the parameters necessary for adjustment to a final formulation of the cell product 90 for storage or for administration to a subject. Appropriate formulation solutions and reagents such as but not limited to human serum albumin, CryoStor™ solution, and antibodies are introduced from the reagent vessels 110 to the formulation chamber 195 in order to produce the final desired cell product 90. The cell product 90 is transferred from the formulation chamber 195 via the product output line 143 to one or more sterile cell product output vessels 196.

In one embodiment of the invention, the process 10 may comprise steps of periodic harvest of cells following the expansion step 60. For example, it may be necessary to monitor cellular expansion or harvest at different times depending upon the nature of the cell culture process being performed within the device 100. In such instances, it is possible to harvest cells periodically from one or more of the expansion surfaces 153 in batches rather than at the same time.

In a further embodiment of the invention, the process 10 may comprise steps of periodic harvest of cells following the expansion step 60, but where the cells are returned from one or more of the expansion surfaces 153 to the cell processing chamber 140 so that additional process steps of analytics 80 and expansion 60 may occur. For example, dependent on the types of cells under culture, if cell growth during the expansion step 60 is slower than expected it may be necessary to replace or replenish spent expansion media. Hence, typically the cartridge 101 comprises additional filtration circuits 170 in fluid communication with the processing chamber 140 so as to allow for repeated filtration and concentration steps to be incorporated into the process 10. These additional steps may be desired by an operator or in an automated manner in response to data collected during any one of the analytics steps 80. Hence, rather than creating unnecessary redundancy within the device 100, inclusion of additional processing, reagents and analytics capacity can facilitate flexibility of the device 100 so that it can handle diverse starting materials of variable quality and quantity.

### Example 3: Comparison of Automated and Manual Process for Producing CAR-T Cells for Use in Cell Therapy

### Overview

To evaluate the possibilities of automated cellular processing, a prototype of the cell processing device according to the specification was built and tested at Celyad. The device was optimized and programmed for the production of CAR-T cells. To assess the performance of the cell processing device, a manual process was run in parallel, and different criteria of the produced cells were compared. As will be detailed below, the automated manufacturing process was successfully tested for the production of CAR-T cells, reaching similar quality parameters as a manual process, including expression of the chimeric antigen and potency. The automated process seems to result in higher cell recovery (day 0-day 2) and higher cell expansion (day 2-day 8). Further improvements that are envisaged are optimization of the cell wash and concentration step to assure higher cell recovery post tangential flow treatment.

### Introduction

Common Chimeric Antigen Receptor T cells (CAR-T) manufacturing processes consist of the following steps:
Enrichment of mononuclear cells
T cell activation
T cells transduction
T cell expansion
T cell formulation

Typically, these steps are performed using specific reagents or vessels. An example of a clinical protocol (e.g. used for the Therapeutic Immunotherapy with NKR-2 (THINK) trial) uses Ficoll for the enrichment of mononuclear cells, flasks for the T cell activation step, bags for the T cell transduction step, a Gas Permeable Rapid Cell Expansion Devices (GREX™, Wilson Wolf®, Saint Paul, Minn.) for T cell expansion, and T cell formulation is done through buffer exchange using a regular centrifuge.

To automate such manufacturing process, the cell processing device prototype relied on similar single use disposables.

### Process Overview

The instant cell processing device prototype (CDP) is an automated fluid handling system, with a touch screen interface for operational control. The CDP consists of peristaltic pumps, valves and a bag mixer for the processing bag. It also includes pressure and fluid sensors. Fluidic movements are managed by the computer-controlled system in the CPS, which synchronizes pumps and valves to achieve the designated task. For ease of reference, the cell processing station (CPS) comprises the computer-controlled system and the touch screen interface, while the cell processing module (CPM) comprises the pumps, valves and bag mixer. The processing bag is part of the fluidic cartridge. Only one CPM was in use for purpose of this comparative test. Further comprised in the CPM is the reagent pack, comprising the disposables for the different process steps. Here, four different single use disposable sets were developed to perform all the required manufacturing steps: one for mononuclear cell enrichment and T cells activation, one for T cells transduction, one for T cell expansion and one for T cells formulation.

Each disposable set comprises inlet/outlet lines that allow for the connection of:
Bags containing reagents and/or cells (part of the reagent pack)
Cell culture vessels (bags or GREX™) (part of the fluidic cartridge)

All connections are performed using sterile welding technology, thereby maintaining the functionally closed nature of the system.

All buffer exchange processes (cell washes, cell concentrations, and reagents exchanges) were performed relying on a tangential flow filtration procedure using hollow fiber filters. Total volume exchange was fixed at 3 times the cell volume during the process and 6 times at the final wash during harvest.

At each manufacturing step, cells (provided either as apheresis product at the start of the process or as the in process cultured cells during the process) are transferred automatically from cell containers (inlet) to the cell processing bag. For cultured cells, the connection from the cell container to the cell processing bag comprises a series of 90° tube elbows and consecutive tubing diameter changes. This particular design aids to disrupt cell clumps (the flow of the cells through such tubes achieves the same effects as gentle up and down pipetting to disrupt cell clumps, but without human intervention) prior the cell processing bag, obtaining a sample consisting of single cells.

The cell processing bag features three sampling bulbs that allow collection of cells for counting, viability and other characterization testing, without compromising the functionally closed nature of the system. A bag mixer guarantees cells concentration homogenization in the processing bag. The processing bag is connected to a scale that allows volume monitoring during the process. For ease of reference, the sampling bulbs and scale are examples of systems used for in situ measurement and testing.

Based on cell counting, number and size of containers downstream (inputs for the CPS), the device can automatically formulate the cells in the right buffer, at the right concentration and perform the appropriate cell seeding. For the prototype, in addition to the processing bag, the fluidic cartridge used in the CPM comprised several vessels of different nature that allow culturing of cells for the prerequisite time. The table below presents vessels used for each step. These vessels are sterile connected to the appropriate disposable sets.

TABLE 3.1
fluidic cartridge vessels used at each step
by the cell processing device prototype

| MANUFACTURING | VESSELS | SEEDING/ |
|---|---|---|
| STEP | USED | VESSEL |
| ACTIVATION | AC 197 bag | 197 ml @ 1 million |
| | (Vuelife ™, | cells/ml |
| | CellGenix ™) | |
| TRANSDUCTION | PL240 bag | 142 ml @ 0.7 million |
| | (OriGen ™) | cells/ml |
| EXPANSION | GREX-100M CS | 1 million/cm² |
| | (Wilson Wolf ®) | |

The seeded culture vessels were maintained at 37° C. and 5% CO₂.

### Experimental Protocol and Results

### Starting Material and Activation (Day 0-Day 2)

A leukapheresis product collected from healthy donors was stored at room temperature and processed within 48 h from collection. Manufacturing process started with the generation of cells for activation by treatment of the leukapheresis product with NH₄Cl (RBCs lysis buffer), as follows:

### Day 0

### Arm 1—CDP

40 mL of leukapheresis product were transferred to the cell processing bag;
The selected volume of leukapheresis was mixed in 3 volume of NH₄Cl (red blood cell lysis buffer);
After 9 minutes at room temperature, the lysis reaction was halted by adding 320 mL of HBSS at RT Buffer exchange was performed with a tangential flow based strategy using a hollow fibre filter (HFF), cells resuspended in X-Vivo™ for counting;
Cells were then formulated in activation buffer and seeded in 4 AC197 VueLife™ bags (Saint Gobain®) at 1M cells/ml for a total of 197 mL/bag.

### Arm 2—Manual

35 ml of leukapheresis product were transferred to the cell processing bag;
The selected volume of leukapheresis was mixed in 3 volume of NH₄Cl (red blood cell lysis buffer);
After 5 minutes at room temperature, the lysis step was stopped by adding 8× volumes of HBSS at RT;
Centrifugation for 10 minutes at 200 g (this step helps in removing platelets);
The supernatant is removed and the cells resuspended in X-Vivo™ for cell counting;
Cells were then formulated in activation buffer and seeded in 6 T175 flasks at 1M cells/ml for a total of 92 ml/flask

Activation buffer comprises 40 ng/ml of soluble anti-human CD3 mAbs (OKT3 clone) and 100 U/ml of IL-2 in X-Vivo™-15 medium supplemented with 5% human AB serum plus 2 mM L-glutamine (complete X-Vivo™-15 medium). The activation step lasts for two days.

Table 3.2 summarizes cell manipulation with NH₄Cl. Cell recovery post NH₄Cl in arm 1 (CDP) was 10% higher than the manual process (77% vs 68%).

Table 3.3 summarizes total cells seeded at activation/vessel type (and number of vessels used), harvested cells at the end of the activation process and cell recovery post activation step. Recovery was 13% higher in the CDP (57% vs 44%)

TABLE 3.2
summary of cell manipulations with NH4Cl in the different arms

| | APHERESIS | WBC | POST NH₄Cl | POST NH₄Cl | POST NH₄Cl |
|---|---|---|---|---|---|
| ARM | USED (ML) | MANIPULATED | RECOVERY | RECOVERY (%) | VIABILITY (%) |
| ARM 1 | 40 | 1.4E+09 | 1.1E+09 | 77% | 88% |
| ARM 2 | 35 | 1.2E+09 | 8.3E+08 | 68% | 92% |

TABLE 3.3
summary of cell activation in the different arms.

| | | | FLASK | HARVESTED | | |
|---|---|---|---|---|---|---|
| | CULTURE | PBMCS | USED- | CELLS POST | | RECOVERY/ |
| | CELL | SEEDED | TYPE AND | ACTIVATION | | SELECTION |
| ARM | ARM | (M) | NUMBER | (M) | VIABILITY | EFFICIENCY |
| ARM 1 | CDP | 7.9E+08 | 4 × AC197 | 4.5E+08 | 88% | 57% |
| ARM 2 | Manual | 5.6E+08 | 6 × T175 | 2.5E+08 | 76% | 44% |
| | control | | | | | |

### Transduction (Day 2-Day 4)

### Day 2

For transduction, activated cells were transduced with retroviral vector in PL240 bags in both arms. As retroviral construct, a CAR-T directed against B7-H6 was used (described in WO2013169691).

A second manual control arm was generated at day 2 starting from cells activated in the CDP: this to evaluate the impact of the CDP driven activation. We will refer to this as Arm 3.

Protocol is as described below:

Transduction was performed with RetroNectin® pre-coated bags.

RetroNectin® pre-coated bags were prepared by overnight coating with RetroNectin® in PBS at the concentration of 8 μg/ml. The day after a blocking step of at least 30 minutes was performed with PBS+HSA 1% at RT.

All the steps described above were performed manually. Bags used with the CDP are sterile connected after the blocking solution was removed.

The vessels used for transduction were cell culture bags, either PL30 or PL240 by Origen™, and with a seeding for PL30/29 ml and PL 240/142 ml. Concentration at seeding was 1M/ml for the manual process and 0.7M/ml for the CDP driven process.

Briefly:

### Arm 1—CDP

Activation bags (AC 197) were sterile connected to the transduction set;
Cells were transferred to the processing bag;
After cell counting, cells were formulated in the transduction media, buffer exchange was performed with a tangential flow based strategy using an HFF;
Cells were then formulated in transduction buffer and seeded in 2 PL240 bags at 0,7M cells/ml for a total of 142 ml/bag.

### Arm 2—Manual

Activated cells were harvested from the T175;
After spinning, cells were resuspended in X-Vivo™, counted and formulated in the full transduction buffer at final concentration of 1×10⁶ cells/ml;
Cells were seeded in 1 PL240 bag at 1M/ml;
   Arm 3—Manual, Starting from CDP Activated Cells

Aiming to further assess and validate the CDP based activation, cells were collected from the remaining cells of arm 1 transduction and manually formulated (with centrifuge) to be seeded in a PL30 bag at concentration of 1M/ml.

Transduction buffer: cells were resuspended in X-Vivo™-15 medium supplemented with 5% human AB serum, 2 mM L-glutamine and transduced by co-culture them with retroviral supernatant (vector) allowing the expression of the chimeric antigen B7H6 and a truncated CD19 (tCD19). Vector represents 50% of the final volume. tCD19 is used to monitor transduction efficiency. Vector used was mTZ47.2.1 (in house name) manufactured by the R&D team of Celyad, with physical particle titer of 1.9·10¹⁰

Additional reagents in the culture media were—at final concentration—100 U/ml of IL-2, 40 ng/ml of anti-CD3.

Table 3.4 summarizes, for each arm, the vessel type use for transduction, vector and titer used, cell harvest and viability and fold of expansion at day 4.

At day 4, the fold expansion of cells manipulated with the CDP was the highest: 2.6 arm 1 vs 1.5 arm 2 or 1.8 arm 3

TABLE 3.4
summary of the transduction step

| | CELLS FOR | | | VESSELS- | CELL | | |
|---|---|---|---|---|---|---|---|
| | TRANSDUCTION | | VECTOR | TYPE AND | HARVESTED | VIABILITY | FOLD |
| ARM | (M) | VECTOR | USE | NUMBER | (m) | (%) | EXPANSION |
| ARM 1 | 2.0E+08 | mTZ47.2.1 | 50% of final | 2 × PL240 | 5.2E+08 | 91% | 2.6 |
| | | | culture volume | | | | |
| ARM 2 | 1.4E+08 | mTZ47.2.1 | 50% of final | 1 × PL240 | 2.2E+08 | 91% | 1.5 |
| | | | culture volume | | | | |
| ARM 3 | 2.9E+07 | mTZ47.2.1 | 50% of final | 1 × PL30 | 5.3E+07 | 86% | 1.8 |
| | | | culture volume | | | | |

### Expansion (Day 4-Day 8)

At day 4, in all arms, expansion of the transduced cells was started by culturing them in a gas permeable cell culture device.

### Arm 1

Post transduction PL240 bags were sterile connected to the reagent bags for expansion, formulated in IL-2 and X-Vivo™. Buffer exchange and volume concentration was performed with a tangential flow based strategy using an HFF.

At the end of the process a G-REX™ 100M-CS (previously connected via sterile connector to the fluidic cartridge) was seeded automatically by the device in 600 ml complete X-Vivo™-15 medium supplemented with 100 U/ml of IL-2.

G-REX™ 100M-CS is a G-REX™ vessel equivalent to the GREX™ 100M. The acronym CS stands for closed system, it implies the presence of pigtails that allow fluid exchange while maintaining the closed nature of the vessel.

### Arm 2 and 3

After transduction T-cells were washed once with HBSS then seeded for the expansion step.

Transduced cells were seeded, at 1×10⁶ cells/cm² of the G-REX™ surface, then into:
Arm 2—G-REX 100M-CS in 600 ml complete X-Vivo™-15 medium supplemented with 100 U/ml of IL-2
Arm 3—G-REX™ 10M in 60 ml complete X-Vivo™-15 medium supplemented with 100 U/ml of IL-2

Approximately 48 hours later, additional 50 percent of fresh complete X-Vivo™-15 plus 100 U/ml of IL-2 was added to each G-REX™ flask. After two more days of culture, cells were harvested at day 8.

### Arm 1

Cells were washed in chilled HBSS and concentrated via a tangential flow strategy using a hollow fiber;
After counting cells were formulated in chilled HBSS by the device at 2,7M/ml, then 300M cells were harvested in a collection bag;
Sample for quality evaluation were taken at the end of the procedure.

Note that, to be able to compare the products in the same way, cryopreserved cell samples were generated by an operator from the collection bag by centrifugation and formulation in a defined cryopreserving buffer. Thus, in the prototype, the harvest was only semi-automatic, while all other steps are managed by the device. This is due to the prototype design, not due to inherent limitations of the device, and resuspension/harvesting of the cells can be done completely automated.

### Arm 2 and 3

Cells were washed once with chilled HBSS using a lab centrifuge. After counting cells, they were formulated for sample collection at the end of the procedure.

Table 3.5 summarizes cell yield at harvest and viability in G-REX™ 10M/G-REX™ 100M/G-REX™ 100M-CS. Fold expansion was 27× for both arm 1 and 2 and 21× for arm 3.

TABLE 3.5
Cell yield at harvest and viability in GREX ™ 10M or GRE ™ 100M

| | CELL | TYPE OF GREX | CELL | | FOLD |
|---|---|---|---|---|---|
| ARM | SEEDED | AND NUMBER | HARVESTED (M) | VIABILITY (%) | EXPANSION |
| ARM 1 | 1.00E+08 | GREX ™ 100M-CS | 2.71E+09 | 96% | 27 |
| ARM 2 | 1.00E+08 | GREX ™ 100M-CS | 2.71E+09 | 97% | 27 |
| ARM 3 | 1.00E+07 | GREX ™ 10M | 2.09E+08 | 97% | 21 |

### Comparability Test Results

During and after the process, samples were taken to assess comparability of the procedures. Comparability was assessed based on:
process parameters
   Cell Count and Viability at each step (recovery or expansion assessment)
   Day 2—cell activation by flow
   Day 4—Vector Copy Number
   Day 4—Expression level of the chimeric antigen receptor
final product characteristics
   Cell Count and Viability
   Expression level of the chimeric antigen receptor
   VCN
   interferon-gamma secretion and killing assay when cultured with cancer cell line
   T cells maturation profile
   T Cell Exhaustion

Note: Cell counting and viability on the CDP was always performed prior the wash and concentration step while for the manual step we always spin and resuspend the cells once

### Test: Cell Activation (Day 2)

Method: flow monitoring of CD69 and CD25 marker

Results:

| | CD25−/ | CD25+/ | CD25+/ | CD25−/ |
|---|---|---|---|---|
| | CD69+ | CD69+ | CD69− | CD69− |
| ARM 1 | 0.38% | 0.38% | 46.5% | 52.7% |
| ARM 2 | 0.38% | 0.37% | 52.2% | 47.1% |
| ARM 3 | 0.30% | 0.32% | 44.1% | 55.3% |
| NOT | 0.35% | 0.054% | 0% | 99.6% |
| ACTIVATED - | | | | |
| PBMCS | | | | |
| NOT | 0.28% | 0.12% | 0% | 99.6% |
| ACTIVATED - | | | | |
| PBMCS | | | | |

Conclusion: CD69 is a marker of early activation while CD25 rises after 24 h. The results indicate that all arms were properly activated and comparable, as indicated by the lack of CD69+ cells and by the shift of about 50% (44%-52%) of the activated arm vs control of the CD25 marker

### Test: Transduction (Day 4)

Method: flow monitoring tCD19 and qPCR evaluating the mean vector copy number (VCN)/cell

Results:

| ARM | VCN | SD | ANTIGEN EXPRESSION |
|---|---|---|---|
| ARM 1 | 1.92 | 0.04 | 40% |
| ARM 2 | 1.92 | 0.06 | 50% |
| ARM 3 | 1.93 | 0.05 | 40% |

Conclusion: Cells of all three arms were successfully transduced, VCN are equivalent in the three arms tested. We were also able to detect tCD19 expression at the end of the transduction phase. 10% more positive tCD19 cells were detected at day 4 for the fully manual condition (50% vs 40%)

### Test: Transduction at the End of the Manufacturing Process (Day 8)

Method: Verified by flow on tCD19 and by qPCR evaluating the vector copy number (VCN)/cell

| ARM | VCN | SD | ANTIGEN EXPRESSION |
|---|---|---|---|
| ARM 1 | 2.43 | 0.07 | 51% |
| ARM 2 | 2.50 | 0.14 | 57% |
| ARM 3 | 2.80 | 0.44 | 53% |

Results:

Conclusion: Cells of all three arms were successfully transduced, VCN equivalence in the three arms is confirmed. tCD19 expression is detectable in all 3 arms ranging between 51% of arm1 (CDP arm) and 57% of arm 2 (Manual arm)

### Test: T Cells Maturation Profile at the End of the Manufacturing Process

Method: Flow monitoring CD45RA and CD62L

Results:

| | CENTRAL | | EFFECTOR | EFFECTOR |
|---|---|---|---|---|
| | MEMORY | NAÏVE | T CELL | MEMORY |
| MATURA- | CD45RA−/ | CD45RA+/ | CD45RA+/ | CD45RA−/ |
| TION | CD62L+ | CD62L+ | CD62L− | CD62L− |
| ARM1 | 45.7% | 7.69% | 4.92% | 41.7% |
| ARM2 | 43.4% | 5.46% | 3.80% | 47.3% |
| ARM3 | 38.0% | 6.49% | 6.41% | 49.1% |

Conclusion: At the end of the manufacturing process most cells presented central memory or effector memory phenotypes, the manufacturing process doesn't impact the T cell profile at the end of the process.

### Test: T Cells Exhaustion at the End of the Manufacturing Process

Method: Flow monitoring PD1 and Lag3

Results

| | PD1−/ | PD1+/ | PD1+/ | PD1−/ |
|---|---|---|---|---|
| | LAG3+ | LAG3+ | LAG3− | LAG3− |
| ARM 1 | 1.97% | 0.94% | 8.02% | 89.1% |
| ARM2 | 1.49% | 1.04% | 10.8% | 86.7% |
| ARM 3 | 1.31% | 1.22% | 14.8% | 82.7% |
| CONTROL | 1.07% | 0.16% | 1.63% | 97.1% |
| PBMCS | | | | |
| CONTROL | 1.26% | 0.27% | 1.87% | 96.6% |
| PBMCS | | | | |

Conclusion: At the end of the manufacturing process most cells (≥80%) are negative for both the exhaustion marker PD1 and LAG3, only 8-14% of the cells were positive for the PD1 marker. Interestingly, the automated process is the one with the lowest expression of PD1.

Control arms are PBMC cells generated at day 0 either by the CDP device or by the manual process, PBMC cells were cryopreserved immediately after the RBCs lysis step.

### Test: T Cells Potency at the End of the Manufacturing Process

Method: CAR-T cytolytic activity (killing assay) on a target cell line (HeLa cells) and IFN-gamma secretion on target cells (K562)

Results:

| | CYTOLYTIC ACTIVITY | | |
|---|---|---|---|
| ARM | % of killed Hela | SD | |
| ARM1 | 85.81% | 3.0% | |
| ARM2 | 98.17% | 0.7% | |
| ARM3 | 96.93% | 0.3% | |
| CONTROL | 34.61% | 7.6% | <LOD |
| CONTROL | 13.29% | 16.9% | <LOD |

LOD: Limit of detection;
LOQ: limit of quantification

IFN GAMMA (ng/mL)

| ARM | Mean | SD |
|---|---|---|
| ARM 1 | 1.79 | 0.03 |
| ARM 2 | 1.91 | 0.12 |
| ARM 3 | >LOQ | >LOQ |
| CONTROL - | <LOD | <LOD |
| PBMCS | | |
| CONTROL - | <LOD | <LOD |
| PBMCS | | |

LOD: Limit of detection;
LOQ: limit of quantification

Conclusion: Upon the exposure to the relevant cancer cell line models, CAR-T cells manufactured in the three arm conditions proved to be equally potent, both in the killing assay and in IFN gamma secretion. Conclusions are qualitative and they are based on a comparison to the PBMCs control.

Control arms are PBMC cells generated at day 0 either by the CDP device or by the manual process, PBMC cells were cryopreserved immediately after the RBCs lysis step.

### CONCLUSION

The CDP proved to be a device suitable to sustain all the manufacturing steps required to manufacture chimeric antigen receptor T cells.

With a cell recovery of 77%, RBC Lysis of a leukapheresis product (instead of a Ficoll procedure) confirmed to be an efficient way to obtain a PBMC-like population. Post activation, the recovery was 57%. These data are in range of the validation data of manufacturing for other CAR-T protocols.

Interestingly, by looking at the count of mononuclear cells, automated processing of the cells using the CDP resulted in higher recovery vs manual methods: 77% vs 68% for the recovery post RBCs Lysis and 57% vs 44% for the recovery post activation.

Viability at day 2 were 88% (automated arm) and 76% (manual arm). These results were lower than what we usually observed in a process that starts with a ficoll selection (typically around 90%). As the product resulted from the RBCs lysis treatment is not as clean as products obtained with a ficoll process, we suspect that the higher cell death observed is due to larger numbers of non-T cells than usually observed with ficoll, these cells will naturally die in the first two days.

Looking at the activation profile at day 2 we observed that cells were properly activated in all arms.

This is in line with earlier internal studies showing that AC bags can sustain activation in a similar fashion than T flasks. In the context of the activation in the CDP evaluation we ended up using a different seeding density during the activation step. 1M/ml was used in all conditions, while the resulting seeding density/cm² was different: 0.5M cells/cm² for the T flask activation vs 0.7 M cells/cm² for the AC bags.

Based on viable cell counting we monitor cell expansion during both transduction (day 2-day 4) and expansion (day 4-day 8) steps.

As summarized in table 3.6 below, viability is always very similar when comparing a fully automated process (arm 1) vs a full manual one (arm 2).

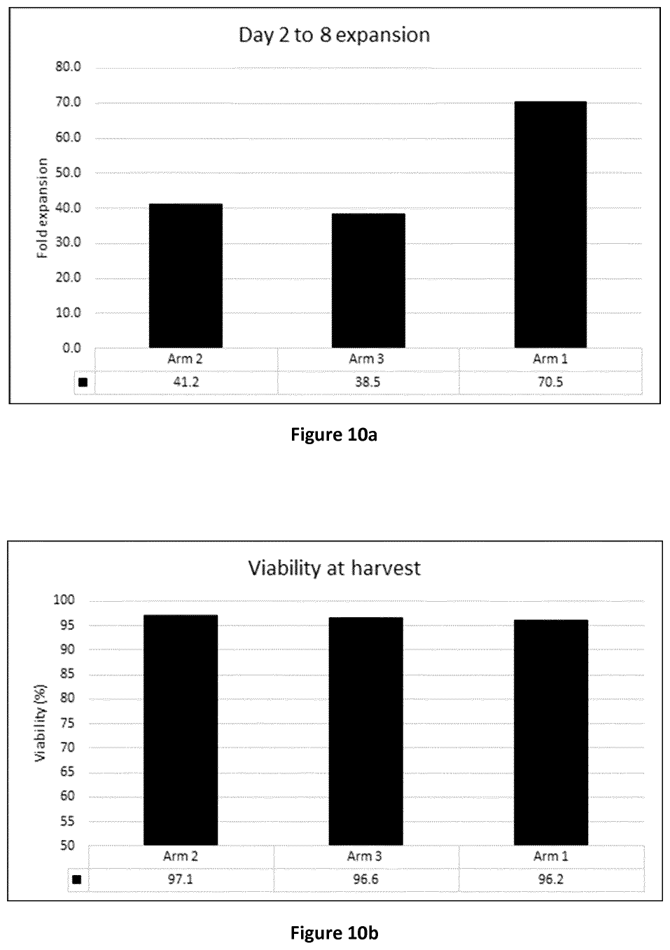

Fold expansion in G-REX™ was the same when comparing these two arms. Since a higher expansion fold was obtained during the transduction in arm 1 (2.6 vs 1.5), the combined expansion is in favor of the automated process (arm 1) vs the fully manual (arm 2): 70.5 vs 41.2 respectively. The combined day 2 to day 8 expansion is also shown in FIG. 10a, the viability at harvest in FIG. 10b.

In the additional control (arm 3) the process was successfully performed; however, we did observe an intermediate expansion during transduction, the difference observed vs arm 1 is probably due to a lower viability (86% vs 90%). During the expansion phase, cells of arm 3 also performed less population doubling when compared to the other two arms. The difference might be due to the use of a different type of GREX™: GREX™ 10M vs GREX™ 100M-CS.

TABLE 3.6
Summary of expansion rate and viability
of CAR-T cells across the different arms.

| | DAY 2-4 | | DAY 4-8 | | |
|---|---|---|---|---|---|
| ARM | Fold Exp. | Viability | Fold Exp. | Viability | COMBINED |
| ARM 1 | 2.6 | 90.5 | 27.1 | 96.2 | 70.5 |
| ARM 2 | 1.5 | 90.5 | 27.1 | 97.1 | 41.2 |
| ARM 3 | 1.8 | 86.1 | 20.9 | 96.6 | 38.5 |

Vector used for the transduction was 50% of the final culture volume used during the transduction step in all 3 arms. In the next generation of disposables, both processing bag size and HFF capabilities will be revisited, allowing the use of vector up to 90% of the transduction volume.

As described, each step of cell washing, concentration, buffer exchange and culture media formulation were performed via tangential flow using hollow fibers (HFF). The process was successfully performed with a total buffer exchange of target of 3 volumes in process and 6 volumes for the final formulation. By evaluating cell recovery pre/post HFF (table 3.7) we identify another possible area of improvement as the recovery ranges between 39% and 86% (see table below). In the past, we did observe that cell aggregation negatively impacts cell recovery. The disposable sets used with the CDP were designed so that while moving cells containing fluid form culture vessels to the processing bag, all cell clumps were disaggregated, resulting in a single cell suspension. Due to the time required to collect the samples and perform a manual count, the cell had started to re-aggregate, resulting in visible clumps within the processing time. We believe that it will be possible to increase the recovery by revisiting disposable design so that cell clumps get disaggregated also prior to the HFF.

TABLE 3.7
Cell recovery rate in the CDP at different stages

| | DAY 0 | DAY 2 | DAY 4 | DAY 8 |
|---|---|---|---|---|
| MANUFACTURING | Activation | Transduction | Expansion | Harvest |
| STEPS | | | | |
| RECOVERY HFF: | N/A | 53% | 86% | 39% |
| AFTER/PRIOR | | | | |

At the end of the manufacturing process all cells were tested for maturation profile, exhaustion and potency. All tests confirmed that the processes used resulted in cells of the same quality: cells are not exhausted and capable of killing cells and produce interferon-gamma in a similar fashion. About 90% of the cells appears to be either central or effector memory T cells, divided approximately evenly.

In conclusion, both the automated process and the manual one allowed the production of CAR-T with similar VCN/cell (range 2.43-2.80) and antigen expression (range 51%-57%). The automated process seems to result in higher cell recovery (day 0-day 2) and higher cell expansion (day 2-day 8) with similar viability of cells at harvest. Further optimization of the cell wash and concentration step is envisaged to assure higher cell recovery post tangential flow treatment.

### Example 4: Independent Concurrent Automated Process for Producing CAR-T Cells for Use in Cell Therapy Run on the Same Device

In Example 3, a comparison was made between automated and manual processing of cells. This confirmed that automated processing using the cell processing device prototype yields similar cell products. Only one cell processing module of the prototype was used for the comparison. In this Example, the automated process as described in Example 3 will be repeated, using two processing modules. These will be operated on the same device, but with a different starting time (however, not in a sequential manner: processing of the second batch will be initiated prior to the first batch being completed. This way, two batches of CAR-T cells will be made in a concurrent and independent way: concurrent, as the processing of both will run on the same device at the same time (or at least, with a significant overlap of time for the process), and independent, as the processing of the second batch can be initiated regardless of the process of the first batch of cells. It is to be understood that while manufacture of both batches of cells will make use of the same computer-controlled system and the touch screen interface, they will rely on individual reagent packs and fluidic cartridges (i.e., each batch is made in its own cell processing module).

It is to be understood that although particular embodiments, specific configurations as well as materials and/or molecules, have been discussed herein for cells and methods according to present invention, various changes or modifications in form and detail may be made without departing from the scope and spirit of this invention. The examples are provided to better illustrate particular embodiments, and they should not be considered limiting the application. The application is limited only by the claims.

1. A cell processing device suitable for performing independent concurrent processing of a plurality of cell preparations, the cell processing device comprising:
(I) a cell processing station; and
(II) a plurality of cell processing modules,
wherein the plurality of cell processing modules engage and communicate with the cell processing station;
wherein each of the plurality of cell processing modules comprises discrete processing compartments defined within, the processing compartments comprising:
i. a reagent pack, the reagent pack comprising one or more reagent vessels; and
ii. one or more fluidic cartridges, each fluidic cartridge comprising one or more cell processing compartments and a cell incubation compartment, and
wherein each of the processing compartments is in fluid communication with each other.

2. The cell processing device of claim 1, wherein the device is configured such that each of the plurality of cell preparations are physically and/or temporally separated throughout processing.

3. The cell processing device of claim 1, wherein each of the plurality of cell processing modules is adapted to process a single cell preparation at once.

4. The cell processing device of claim 1, wherein the cell processing module comprises a closed, substantially aseptic environment for cell processing.

5. The cell processing device of claim 4, wherein the closed, substantially aseptic environment is provided within the reagent pack and/or the fluidic cartridge.

6. The cell processing device of claim 1, wherein at least part of the cell processing module is a single-use component.

7. The cell processing device of claim 6, wherein substantially all of the cell processing module is a single-use component.

8. The cell processing device of claim 7, wherein the single use component of the cell processing module is selected from the group consisting of: the fluidic cartridge; the reagent pack; and both the fluidic cartridge and the reagent pack.

9. The cell processing device of claim 1, wherein the cell processing station comprises centralised facilities that may be used by one or more of the plurality of cell processing modules.

10. The cell processing device of claim 9, wherein the centralised facilities are selected from the group consisting of:
i. a platform chassis;
ii. a user interface display;
iii. software and operating system licenses;
iv. a central processing unit (CPU);
v. a main control system including embedded controller and Program Logic Controller (PLC);
vi. a power supply and power distribution system;
vii. thermal management equipment required for controlling the temperature of the cell processing module or parts thereof;
viii. an incubator gas mixture supply facility;
ix. one or more systems used for in situ measurement and/or testing; and
x. a centrifugation drive system.

11. The cell processing device of claim 10, wherein the in situ measurement and/or testing is selected from the group consisting of: cell counting; cell identification; purity; homogeneity; potency; characterisation; and quality control.

12. The cell processing device of any one of claims 1 to 11, wherein the cell processing module is adapted to contain the cell preparation throughout cell processing.

13. The cell processing device of claim 1, wherein each of the plurality of cell processing modules comprises one or more connectors suitable for connection with the centralised facilities on the cell processing system.

14-21. (canceled)

22. A fluidic cartridge suitable for providing a closed aseptic environment for cell processing therein, wherein the fluidic cartridge comprises at least one input port, a separation chamber, an activation chamber, a transduction chamber, a cell culture chamber, and at least one output port.

23. The fluidic cartridge of claim 22, wherein the fluidic cartridge further comprises elements selected from the group consisting of:
i. Filters, centrifuges and other active surfaces for separating the cell preparation into different components;
ii. Reaction vessels, flasks and beakers for incubating cells and introducing active reagents; and
iii. Galleries, channels and fluidic circuits to direct the flow of fluids around, into and out of the fluidic cartridge.

24. The fluidic cartridge of claim 22, wherein the fluidic cartridge further comprises at least one pump and at least one valve to at least partially control fluid flow, wherein fluid flow is comprises flow of fluid through a fluid circuitry in the fluidic cartridge, flow into the fluidic cartridge, and flow out of the fluidic cartridge.

25. The fluidic cartridge of claim 24, wherein the at least one pump is selected from the group consisting of: a positive displacement pump, a diaphragm, a plunger-style pump, impeller pumps, peristaltic pumps and a combination thereof; and wherein the valve is selected from the group consisting of a diaphragm valve, a rotating valve; and a combination thereof.

26-30. (canceled)

31. A cell processing module suitable for integration with a cell processing station, wherein the cell processing module comprises:
i. a cell processing unit, the cell processing unit comprising a fluidic cartridge and a reagent pack, wherein the fluidic cartridge comprises at least one input port, a separation chamber, an activation chamber, a transduction chamber, a cell culture chamber, and at least one output port, and wherein the reagent pack comprises one or more reagent vessels, wherein the one or more reagent vessels in the reagent pack are adapted to contain one or more non-cellular fluids required for cell processing; and
ii. a means to retain and engage the fluidic cartridge and the reagent pack such that fluidic connection points of the fluidic cartridge and the reagent pack couple to provide fluid communication between the fluidic cartridge and the reagent pack.

32. The cell processing module of claim 31, wherein the cell processing module further comprises one or more of:
at least one connector for connection of the cell processing module or components thereof to the cell processing station;
at least one sensor;
an incubator enclosure to provide a temperature controlled environment to the cell processing module;
a heater pad and controller to provide thermal energy to the cell processing module;
one or more mechanical actuators housed within the cell processing module to actuate elements within the fluidic cartridge;
one or more mechanical actuators housed within the cell processing module to actuate elements within the reagent pack;
a centrifugal facility.

33. The cell processing module of claim 32, wherein when present, the at least one sensor is selected from the group consisting of thermocouples/thermistors to measure temperature; pressure transducers to measure fluid pressure; flow meters to measure fluid flow rate; and biosensors as required by the process.

34-50. (canceled)
